(19) United States
(12) Patent Application Publication
(10) Pub. No.: US 2024/0285644 A1
(43) Pub. Date: Aug. 29, 2024

(54) ASSESSING AND TREATING PROSTATE CANCER

(71) Applicant: Mayo Foundation for Medical Education and Research, Rochester, MN (US)

(72) Inventors: Roman Thaler, Rochester, MN (US); Andre J. van Wijnen, Rochester, MN (US); Fabrice Lucien-Matteoni, Rochester, MN (US); Haidong Dong, Rochester, MN (US); Haojie Huang, Rochester, MN (US); Sean S. Park, Rochester, MN (US); Jacob J. Orme, Rochester, MN (US)

(21) Appl. No.: 18/570,491

(22) PCT Filed: Jun. 17, 2022
(86) PCT No.: PCT/US2022/033934

§ 371 Date: Dec. 14, 2023

Related U.S. Application Data
(60) Provisional application No. 63/211,802, filed on Jun. 17, 2021.

(51) Int. Cl. A61K 31/55; A61K 31/416; A61K 31/4545; A61K 31/4709; A61K 31/502; A61K 31/5025; A61K 31/517; A61K 31/5377; A61K 38/07; A61K 45/06; A61P 35/00; C07K 16/28; C07K 16/32

(52) U.S. Cl. CPC A61K 31/55; A61K 31/416; A61K 31/4545; A61K 31/4709; A61K 31/502; A61K 31/5025; A61K 31/517; A61K 31/5377; A61K 38/07; A61K 45/06; A61P 35/00; C07K 16/2896; C07K 16/32

(57) ABSTRACT

This document provides methods and materials for assessing and/or treating mammals (e.g., humans) having prostate cancer. In some cases, methods and materials for identifying a mammal (e.g., a human) as having a resistant prostate cancer (e.g., a prostate cancer that it has become resistant or refractory to one or more cancer treatments) are provided. In some cases, methods and materials for treating a mammal (e.g., a human) having prostate cancer (e.g., a resistant prostate cancer) are provided.

### CROSS REFERENCE TO RELATED APPLICATIONS

This application claims the benefit of U.S. Provisional Application Ser. No. 63/211,802, filed on Jun. 17, 2021, which is incorporated herein by reference in its entirety.

### BACKGROUND

### 1. Technical Field

This document relates to methods and materials for assessing and/or treating mammals (e.g., humans) having prostate cancer. In some cases, the methods and materials provided herein can be used to identify a mammal (e.g., a human) as having a treatment-resistant prostate cancer (e.g., a prostate cancer that it has become resistant or refractory to one or more cancer treatments). In some cases, the methods and materials provided herein can be used to treat a mammal (e.g., a human) having prostate cancer (e.g., a treatment-resistant prostate cancer).

### 2. Background Information

Androgen dependence or independence has been the prevailing paradigm for clinical treatment of prostate cancer for the last seventy-five years (Huggins, J. Am. Med. Assoc., 131:576-81 (1946); and Feldman et al., Nat. Rev. Cancer, 1:34-45 (2001)). Prostate adenocarcinomas that respond to androgen deprivation therapy (ADT) generally express normal androgen receptor (AR) and are less likely to metastasize than the aggressive castration-resistant prostate cancers (CRPC) (Taplin et al., N. Engl. J. Med. 332:1393-8 (1995)). Unfortunately, all recurrent metastatic castration sensitive prostate cancer (CSPC) eventually progress to CRPC (Ross et al., Cancer, 112:1247-53 (2008)). Improved approaches to ADT resistance have relied on the development of increasingly potent oral second-generation antiandrogens like enzalutamide and abiraterone. Unfortunately, CRPC is associated with poor clinical outcomes as the disease becomes progressively resistant to second-generation antiandrogens and chemotherapy (Siegel et al., CA Cancer J. Clin., 70:7-30 (2020)).

### SUMMARY

This document relates to methods and materials for assessing and/or treating mammals (e.g., humans) having prostate cancer. In some cases, the methods and materials provided herein can be used to identify a mammal (e.g., a human) as having a metastatic prostate cancer. For example, the presence or absence of an elevated level of a neuregulin 1 (NRG-1) polypeptide in a sample (e.g., a blood sample) obtained from a mammal (e.g., a human) having prostate cancer can be used to determine whether a prostate cancer has metastasized. In some cases, the methods and materials provided herein can be used to identify a mammal (e.g., a human) as having a treatment-resistant prostate cancer (e.g., a prostate cancer that it has become resistant or refractory to one or more cancer treatments). For example, the presence or absence of an elevated level of a NRG-1 polypeptide in a sample (e.g., a blood sample) obtained from a mammal (e.g., a human) having prostate cancer can be used to determine whether a prostate cancer has progressed from a CSPC (which can also be referred to as a hormone-sensitive prostate cancer (HSCP), castration naïve prostate cancer (CNPC), or a treatment-sensitive prostate cancer) to a treatment-resistant prostate cancer (e.g., a prostate cancer that it has become resistant or refractory to one or more cancer treatments). In some cases, a treatment-resistant prostate cancer can be resistant to one or more cancer treatments (e.g., androgen deprivation therapy, anti-androgen therapy, chemotherapy, radiotherapy, or other treatment modalities). For example, a treatment-resistant prostate cancer can be a CRPC (which can also be referred to as a hormone-resistant prostate cancer (HRPC)). In some cases, the methods and materials provided herein can be used to treat a mammal (e.g., a human) having prostate cancer (e.g., CRPC). For example, one or more inhibitors of a NRG-1 polypeptide can be administered to a mammal (e.g., a human) having CRPC to restore treatment sensitivity (e.g., androgen sensitivity) to the CRPC, and, optionally, one or more anti-androgen agents can be administered to that mammal to treat the mammal.

NRG-1 polypeptides are transmembrane polypeptides that can be cleaved such that a soluble form of a NRG-1 polypeptide (a sNRG-1 polypeptide) can be used as a systemic biomarker that can be detected in circulating blood. As demonstrated herein, the presence or absence of an elevated level of a NRG-1 polypeptide (e.g., a sNRG-1 polypeptide) in a sample (e.g., a blood sample) obtained from a mammal (e.g., a human) having prostate cancer can be used to determine whether or not a prostate cancer has progressed from a CSPC to a treatment-resistant prostate cancer (e.g., a prostate cancer that it has become resistant or refractory to one or more cancer treatments). For example, the presence of an elevated level of a NRG-1 polypeptide can be detected in the blood of a mammal (e.g., a human) having CRPC. Also as demonstrated herein, NRG-1 polypeptide signaling can be mediated by androgen resistance in CRPCs, and can be targeted to restore androgen sensitivity to CRPCs. For example, one or more inhibitors of a NRG-1 polypeptide can be administered to a mammal (e.g., a human) having CRPC to restore androgen sensitivity to the CRPC, and, optionally, one or more anti-androgen agents can be administered to the mammal to treat the mammal.

Having the ability to identify a mammal having prostate cancer as having a CRPC as described herein (e.g., based on the presence of an elevated level of a NRG-1 polypeptide in a sample (e.g., a blood sample) obtained from the mammal) provides a simple and non-invasive method for diagnosing treatment-resistant prostate cancers (e.g., CRPCs). In addition, the ability to restore treatment sensitivity (e.g., androgen sensitivity) to CRPCs allows patients and clinicians to overcome anti-androgen resistance and to treat CRPCs.

In general, one aspect of this document features methods for assessing a mammal having prostate cancer. The methods can include, or consist essentially of, (a) detecting an elevated level of a NRG-1 polypeptide in a blood sample from the mammal; (b) classifying the mammal as having a CRPC if the presence of the elevated level is detected; and (c) classifying the mammal as not having the CRPC if the absence of the elevated level is detected. The mammal can be a human. The blood sample can be plasma. The elevated can include at least 3 nanograms of the NRG-1 polypeptide per milliliter of the blood sample (ng/mL). The method can include detecting the presence of the elevated level of the polypeptide. The method can include classifying the mammal as having the CRPC prostate cancer. The method can include detecting the absence of the elevated level of the polypeptide. The method can include classifying the mammal as not having the CRPC prostate cancer. The prostate cancer can be a metastatic prostate cancer.

In another aspect, this document features methods for restoring androgen sensitivity to a CRPC within a mammal. The methods can include, or consist essentially of, subjecting a mammal having a CRPC to a therapy that reduces a systemic level of a NRG-1 polypeptide within the mammal. The mammal can be a human. The therapy can be radiotherapy, cryoablation, radiofrequency ablation, microwave ablation, surgery, metastases-directed stereotactic body radiotherapy (SBRT), or therapeutic plasma exchange (TPE).

In another aspect, this document features methods for restoring androgen sensitivity to a CRPC within a mammal. The methods can include, or consist essentially of, administering an inhibitor of a NRG-1 polypeptide to a mammal having a CRPC. The mammal can be a human. The method can be effective to reduce a systemic level of a NRG-1 polypeptide within the mammal. The inhibitor of the NRG-1 polypeptide can be an inhibitor of NRG-1 polypeptide activity. The inhibitor of the NRG-1 polypeptide can be an inhibitor of NRG-1 polypeptide expression. The inhibitor of the NRG-1 polypeptide can be rucaparib, olaparib, sorafenib, or TAPI-2. The CRPC can be a metastatic CRPC.

In another aspect, this document features methods for restoring androgen sensitivity to a CRPC within a mammal. The methods can include, or consist essentially of, administering an inhibitor of a disintegrin and metalloproteinase domain-containing protein (ADAM) 10 polypeptide to a mammal having a CRPC. The mammal can be a human. The can be effective to reduce a systemic level of a NRG-1 polypeptide within the mammal. The inhibitor of the ADAM10 polypeptide can be an inhibitor of ADAM10 polypeptide activity. The inhibitor of the ADAM10 polypeptide can be an inhibitor of ADAM10 polypeptide expression. The inhibitor of the NRG-1 polypeptide can be INCB8765, GI 254023X, TAPI-0, or TAPI-2. The CRPC can be a metastatic CRPC.

In another aspect, this document features methods for restoring androgen sensitivity to a CRPC within a mammal. The methods can include, or consist essentially of, administering an inhibitor of an ADAM17 polypeptide to a mammal having a CRPC. The mammal can be a human. The method can be effective to reduce a systemic level of a NRG-1 polypeptide within the mammal. The inhibitor of the ADAM17 polypeptide can be an inhibitor of ADAM17 polypeptide activity. The inhibitor of the ADAM17 polypeptide can be an inhibitor of ADAM17 polypeptide expression. The inhibitor of the NRG-1 polypeptide can be an anti-ADAM17 D1(A12) antibody, TAPI-0, or TAPI-2. The CRPC can be a metastatic CRPC.

In another aspect, this document features methods for restoring androgen sensitivity to a CRPC within a mammal. The methods can include, or consist essentially of, administering an inhibitor of a poly (ADP-ribose) polymerase (PARP) polypeptide to a mammal having a CRPC. The mammal can be a human. The method can be effective to reduce a systemic level of a NRG-1 polypeptide within the mammal. The inhibitor of the PARP polypeptide can be an inhibitor of PARP polypeptide activity. The inhibitor of the PARP polypeptide is can be inhibitor of PARP polypeptide expression. The inhibitor of the NRG-1 polypeptide can be olaparib, rucaparib, niraparib, or talazoparib. The CRPC can be a metastatic CRPC.

In another aspect, this document features methods for restoring androgen sensitivity to a CRPC within a mammal. The methods can include, or consist essentially of, administering an agent that can inhibit heterodimerization of a human epidermal growth factor receptor (HER) 2 polypeptide and a HER3 polypeptide (HER2/HER3 heterodimerization) within a mammal having a CRPC. The mammal can be a human. The agent can induce homodimerization of two HER3 polypeptides (HER3 homodimerization). The agent can be trastuzumab, ARRY-380, erlotinib, gefitinib, afatinib, neratinib, or pertuzumab. The CRPC can be a metastatic CRPC.

In another aspect, this document features methods for treating a mammal having CRPC. The methods can include, or consist essentially of, (a) subjecting a mammal having a CRPC to a therapy that can reduce a systemic level of NRG-1 polypeptides within the mammal; and (b) administering an anti-androgen agent to the mammal. The mammal can be a human. The therapy can be metastases-directed SBRT or TPE. The anti-androgen agent can be leuprolide, goserelin, triptorelin, histrelin, degarelix, abiraterone, ketoconazole, flutamide, bicalutamide, nilutamide, enzalutamide, apalutamide, darolutamide, or bicalutamide. The CRPC can be a metastatic CRPC.

In another aspect, this document features methods for treating a mammal having CRPC. The methods can include, or consist essentially of, (a) administering an inhibitor of a NRG-1 polypeptide to a mammal having a CRPC; and (b) administering an anti-androgen agent to said mammal. The mammal can be a human. The inhibitor of the NRG-1 polypeptide can be an inhibitor of NRG-1 polypeptide activity. The inhibitor of the NRG-1 polypeptide can be an inhibitor of NRG-1 polypeptide expression. The inhibitor of the NRG-1 polypeptide can be rucaparib, olaparib, sorafenib, or TAPI-2. The CRPC can be a metastatic CRPC. The anti-androgen agent can be leuprolide, goserelin, triptorelin, histrelin, degarelix, abiraterone, ketoconazole, flutamide, bicalutamide, nilutamide, enzalutamide, apalutamide, darolutamide, or bicalutamide.

In another aspect, this document features methods for treating a mammal having CRPC. The methods can include, or consist essentially of, (a) administering an inhibitor of an ADAM10 polypeptide to a mammal having a CRPC; and (b) administering an anti-androgen agent to the mammal. The mammal can be a human. The inhibitor of the ADAM10 polypeptide can be an inhibitor of ADAM10 polypeptide activity. The inhibitor of the ADAM10 polypeptide can be an inhibitor of ADAM10 polypeptide expression. The inhibitor of the ADAM10 polypeptide can be INCB8765, GI 254023X, TAPI-0, or TAPI-2.

The CRPC can be a metastatic CRPC. The anti-androgen agent can be leuprolide, goserelin, triptorelin, histrelin, degarelix, abiraterone, ketoconazole, flutamide, bicalutamide, nilutamide, enzalutamide, apalutamide, darolutamide, or bicalutamide.

In another aspect, this document features methods for treating a mammal having CRPC. The methods can include, or consist essentially of, (a) administering an inhibitor of an ADAM17 polypeptide to a mammal having a CRPC; and (b) administering an anti-androgen agent to the mammal. The mammal can be a human. The inhibitor of the ADAM17 polypeptide can be an inhibitor of ADAM17 polypeptide activity. The inhibitor of the ADAM17 polypeptide can be an inhibitor of ADAM17 polypeptide expression. The inhibitor of the ADAM17 polypeptide can be an anti-ADAM17 D1(A12) antibody, TAPI-0, or TAPI-2. The CRPC can be a metastatic CRPC. The anti-androgen agent can be leuprolide, goserelin, triptorelin, histrelin, degarelix, abiraterone, ketoconazole, flutamide, bicalutamide, nilutamide, enzalutamide, apalutamide, darolutamide, or bicalutamide.

In another aspect, this document features methods for treating a mammal having CRPC. The methods can include, or consist essentially of, (a) administering an inhibitor of a PARP polypeptide to a mammal having a CRPC; and (b) administering an anti-androgen agent to said mammal. The mammal can be a human. The inhibitor of the PARP polypeptide can be an inhibitor of PARP polypeptide activity. The inhibitor of the PARP polypeptide can be an inhibitor of PARP polypeptide expression. The inhibitor of the PARP polypeptide can be olaparib, rucaparib, niraparib, or talazoparib. The CRPC can be a metastatic CRPC. The anti-androgen agent can be leuprolide, goserelin, triptorelin, histrelin, degarelix, abiraterone, ketoconazole, flutamide, bicalutamide, nilutamide, enzalutamide, apalutamide, darolutamide, or bicalutamide.

In another aspect, this document features methods for treating a mammal having CRPC. The methods can include, or consist essentially of, (a) administering an agent that can inhibit HER2/HER3 heterodimerization within a mammal having a CRPC; and (b) administering an anti-androgen agent to the mammal. The mammal can be a human. The agent can induce HER3 homodimerization. The agent can be trastuzumab, ARRY-380, erlotinib, gefitinib, afatinib, neratinib, or pertuzumab. The CRPC can be a metastatic CRPC. The anti-androgen agent can be leuprolide, goserelin, triptorelin, histrelin, degarelix, abiraterone, ketoconazole, flutamide, bicalutamide, nilutamide, enzalutamide, apalutamide, darolutamide, or bicalutamide.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1L are plots showing that NRG-1 is a systemic biomarker in prostate cancer that switches from CSPC to CRPC. FIGS. 1A-1B are plots showing that patients with very high NRG-1 transcripts in the tumor environment in the TCGA cohort versus the SU2C cohort experience paradoxically superior and inferior outcomes, respectively. FIGS. 1C-1D are plots showing that patients with oligometastatic CRPC exhibited significantly elevated NRG-1 levels (mean 4.89 ng/ml, 95% CI 4.49-5.28 ng/mL) versus healthy controls (mean 3.2 ng/mL, 95% CI 2.84-3.55 ng/mL, p<0.0001) or patients with oligometastatic CSPC (mean 3.39 ng/ml, 95% CI 2.67-4.12 ng/mL, p<0.0001). FIGS. 1E-1H are plots showing that patients with oligometastatic CSPC and elevated sNRG-1 levels experience superior overall survival (p=0.045) and distant progression-free survival (p=0.0019) with a trend toward improved biochemical progression-free survival. FIGS. 1I-1L are plots showing that patients with oligometastatic CRPC and elevated sNRG-1 levels experience inferior local (p=0.001), distant (p=0.0036), and biochemical (p=0.045) progression-free survival with a trend toward worsened overall survival.

FIGS. 2A-2B are plots showing that NRG-1 is a systemic biomarker in prostate cancer that switches from CSPC to CRPC. FIG. 2A is a plot of survival probability over time of patients with high NRG-1 transcripts in the tumor environment of the TCGA cohort versus the SUC2C cohort. FIG. 2B is a plot to determine sensitivity and specificity of GRG-1 levels predicting patient outcomes.

FIGS. 3A-3F. FIG. 3A is a depiction of a western blot of three different cell lines for androgen receptor (AR). Cell lines Du145 and PC3 are antiandrogen-resistant whereas cell line LNCaP is antiandrogen-sensitive. FIGS. 3B-3F are plots of survival of patients from the SU2C cohort with various ratios of HER2, HER3, HER4, and EGFR.

FIGS. 4A-4F are plots showing that NRG-1 plays paradoxical roles in CSPC and CRPC. FIG. 4A is a plot of treatment with enzalutamide (Enz, 3 μM) and rhNRG-1 of antiandrogen-sensitive LNCaP cells. Treatments function in an additive manner. FIGS. 4B-4C are plots of NRG-1 treatment of antiandrogen-resistant Du145 and PC3 cells. Cells are rescued from enzalutamide toxicity. FIG. 4D is a plot of rhNRG-1 and radiation treatment of LNCaP cells. Cells are killed by radiation. FIGS. 4E-4F are plots of rhNRG-1 and radiation treatment of treatment Du145 and PC3 cells. Cells are rescued from radiation-induced cell death.

FIGS. 5A-5G are plots showing that prostate cancer cells induce MSCs to produce NRG-1 in a PARPi- and ADAMs-dependent manner. FIGS. 5A-5B are plots of an analysis of a healthy whole human single-cell transcriptome (GSE159929) showing that monocytes and mesenchymal cells express NRG-1. FIGS. 5C-5D are plots of an analysis of CRPC tumor bed single-cell transcriptome (GSE141445) showing that monocytes and mesenchymal cells express NRG-1 in the tumor microenvironment. FIG. 5E is a plot showing that supernatants from LNCaP and PC3 cells induce increased NRG-1 transcription in mesenchymal stem cells (MSCs). FIG. 5F is a plot showing that inhibition of ADAM10/ADAM17 or PARP reduces MSC NRG-1 production. FIG. 5G is a plot showing that patients with CRPC undergoing oligometastatic site radiotherapy experience a significant decrease in sNRG-1 levels at day 14 after radiation.

FIG. 6A is a plot of survival of MSC cells with TAPI-2, olaparib, or talazoparib treatment. FIG. 6B is a plot of relative NRG-1 expression following various levels of radiation. FIG. 6C is a plot of NRG-1 expression post-radiation in CSPC cancer pre-SBRT and 14 days post-SBRT. FIG. 6D is a set of graphs of overall, local PFS (progression free survival), distant PFS, and biochemical PFS survival.

FIG. 7A-7E are plots showing that a NRG-1 molecular switch in prostate cancer is mediated by tumor cell surface HER2 and HER3. FIG. 7A is a western blot showing that antiandrogen-sensitive LNCaP cells express high levels of HER3 and low levels of HER2 relative to antiandrogen-resistant Du145 and PC3 cells. FIG. 7B is a plot showing that high tumor HER2/HER3 ratios predict poor outcomes in a CRPC cohort. FIGS. 7C-7D are plots showing that overexpression of HER2 or knockdown of shHER3 rescues LNCaP cells from antiandrogen enzalutamide and NRG-1. FIG. 7E is a plot showing that lapatinib-induced HER2/HER3 dimerization rescues LNCaP cells from antiandrogen enzalutamide and NRG-1. FIG. 7F is an exemplary model depicting the NRG-1 switch in prostate cancer, and that HER3 homodimerization leads to prostate cell death, whereas HER2/HER3 dimerization leads to prostate cell treatment resistance.

FIG. 8A is a plot of the knock down of RNA transcripts of LNCaP cells transfected with shControl, shHER3-A, shHER3-B, or shHER3-C RNA. FIG. 8B is a plot of the survival of LNCaP cells treated with shControl, shHER3-A, shHER3-B, or shHER3-C RNA in combination with enzalutamide only or enzalutamide+NRG-1 (50 μg/mL) treatment.

FIG. 8C are plots of the knock down of RNA transcripts of Du145 cells transfected with shControl, shHER3-A, shHER3-B, or shHER3-C RNA and of the survival of Du145 cells treated with shControl, shHER3-A, shHER3-B, or shHER3-C RNA in combination with vehicle or NRG-1 (50 μg/mL) treatment. FIG. 8D are plots of the knock down of RNA transcripts of Du145 cells transfected with shControl, shHER4-A, shHER4-B, or shHER4-C RNA and of the survival of Du145 cells treated with shControl, shHER4-A, shHER4-B, or shHER4-C RNA in combination with vehicle or NRG-1 (50 μg/mL) treatment.

FIG. 9 is a plot of the blood levels of two different patients (001 and 002) NRG-1 before (Day 0) and after (Days 7, 14) treatment with a PARP inhibitor (001 with niraparib, 002 with Olaparib). Blood levels of NRG-1 are reduced in both patients following administration of a PARP inhibitor.

FIG. 10 is a plot showing that C4-2-Con cells, which are still semi-CSPC, were killed by NRG-1.

FIGS. 11A-11C are plots showing that NRG-1 increases survival in castration-resistant prostate cancer cells DU145, PC3 and C4-2-EnZ-R.

FIG. 12 is a plot showing that NRG-1 was produced by mesenchymal cells in patients with prostate cancer.

FIG. 13 is a plot showing that PARP inhibitors significantly reduced systemic NRG-1 in patients treated with these compounds.

FIG. 14 is a plot showing that isolated prostate cancer cell-derived extracellular vesicles (P-EVs)—but not heat-killed EVs or EV-depleted supernatants—induced mesenchymal cell (MC) NRG-1 production.

FIGS. 15A and 15B. FIG. 15A is plot showing that patient-derived xenografts (PDX) showed detectable systemic NRG-1. FIG. 15B is a schematic of an experimental design for an in vivo xenograft study. Animals are implanted with a PDX and then treated with vehicle, PARPi, enzalutamide (ENZ), or PARPi in combination with enzalutamide (PARPi+ENZ).

### DETAILED DESCRIPTION

This document provides methods and materials for assessing and/or treating mammals (e.g., humans) having prostate cancer. In some cases, the methods and materials provided herein can be used to identify a mammal (e.g., a human) as having a metastatic prostate cancer. For example, the presence or absence of an elevated level of a NRG-1 polypeptide in a sample (e.g., a blood sample) obtained from a mammal (e.g., a human) having prostate cancer can be used to determine whether a prostate cancer has metastasized.

In some cases, the methods and materials provided herein can be used to identify a mammal (e.g., a human) as having a treatment-resistant prostate cancer (e.g., a prostate cancer that it has become resistant or refractory to one or more cancer treatments). For example, the presence or absence of an elevated level of a NRG-1 polypeptide in a sample (e.g., a blood sample) obtained from a mammal (e.g., a human) having prostate cancer can be used to determine whether a prostate cancer has progressed from a CSPC to a treatment-resistant prostate cancer (e.g., a prostate cancer that it has become resistant or refractory to one or more cancer treatments). In some cases, the methods and materials provided herein can be used to treat a mammal (e.g., a human) having prostate cancer (e.g., CRPC). For example, one or more inhibitors of a NRG-1 polypeptide can be administered to a mammal (e.g., a human) having CRPC to restore treatment sensitivity (e.g., androgen sensitivity) to the CRPC, and, optionally, one or more anti-androgen agents can be administered to the mammal to treat the mammal.

The term “elevated level” as used herein with respect to a level of a NRG-1 polypeptide (e.g., a sNRG-1 polypeptide) refers to any level that is greater than a reference level of the NRG-1 polypeptide (e.g., the sNRG-1 polypeptide). The term “reference level” as used herein with respect to a NRG-1 polypeptide (e.g., a sNRG-1 polypeptide) refers to the level of the NRG-1 polypeptide (e.g., the sNRG-1 polypeptide) typically observed in a sample (e.g., a control sample) from one or more mammals (e.g., humans) without metastatic prostate cancer. Control samples can include, without limitation, samples from normal (e.g., healthy) mammals, non-cancerous cells (e.g., non-cancerous primary cells and non-cancerous cells lines), and mesenchymal stem cells (MSCs). In some cases, an elevated level of a NRG-1 polypeptide (e.g., a sNRG-1 polypeptide) can be a level that is at least 2 (e.g., at least 5, at least 10, at least 15, at least 20, at least 25, at least 35, or at least 50) fold greater relative to a reference level of the NRG-1 polypeptide (e.g., the sNRG-1 polypeptide). In some cases, an elevated level of a NRG-1 polypeptide (e.g., a sNRG-1 polypeptide) can be a level that is at least 3 nanograms of the NRG-1 polypeptide (e.g., the sNRG-1 polypeptide) per milliliter (ng/ml) of sample (e.g., plasma). For example, an elevated level of a NRG-1 polypeptide (e.g., a sNRG-1 polypeptide) can be a level that is from about 3 ng/ml to about 15 ng/ml of the NRG-1 polypeptide (e.g., the sNRG-1 polypeptide). In some cases, when control samples have undetectable levels of a NRG-1 polypeptide (e.g., a sNRG-1 polypeptide), an elevated level can be a detectable level of the NRG-1 polypeptide (e.g., the sNRG-1 polypeptide). It will be appreciated that levels from comparable samples are used when determining whether or not a particular level is an elevated level.

Any appropriate mammal having prostate cancer can be assessed and/or treated as described herein. Examples of mammals that can have prostate cancer and can be assessed and/or treated as described herein include, without limitation, humans, non-human primates (e.g., monkeys), dogs, cats, horses, cows, pigs, sheep, mice, and rats. In some cases, a mammal can be a male mammal. For example, a male human having prostate cancer can be assessed and/or treated as described herein.

Any appropriate sample can be assessed to determine if a mammal (e.g., a human) has an elevated level of a NRG-1 polypeptide (e.g., a sNRG-1 polypeptide). In some cases, a sample can be a biological sample. In some cases, a sample can contain one or more biological molecules (e.g., nucleic acids such as DNA and RNA, polypeptides, carbohydrates, lipids, hormones, and/or metabolites). For example, biological samples such as fluid (e.g., whole blood, peripheral blood, serum, plasma, urine, or cell suspensions) samples and tissue (e.g., prostate tissue and metastatic site tissue) samples can be obtained from a mammal and assessed for the presence of an elevated level of NRG-1 polypeptides (e.g., sNRG-1 polypeptides) and/or the human epidermal growth factor receptor (HER) status (e.g., the presence of HER2/HER3 heterodimeric receptors or the presence of HER3 homodimeric receptors). A sample can be a fresh sample or a fixed sample (e.g., a formaldehyde-fixed sample or a formalin-fixed sample). In some cases, a sample can be a processed sample (e.g., an embedded sample such as a paraffin or OCT embedded sample). In some cases, one or more biological molecules can be isolated from a sample. For example, nucleic acid (e.g., RNA such as messenger RNA (mRNA)) can be isolated from a sample and can be assessed as described herein. For example, one or more polypeptides can be isolated from a sample and can be assessed as described herein. In some cases, a peripheral blood sample can be obtained and assessed to determine whether or not the mammal has an elevated level of a NRG-1 polypeptide (e.g., a sNRG-1 polypeptide). In some cases, plasma can be obtained and assessed to determine whether or not a mammal has an elevated level of a NRG-1 polypeptide (e.g., a sNRG-1 polypeptide).

A sample (e.g., a blood sample) obtained from a mammal (e.g., a human) having prostate cancer can be assessed for any appropriate NRG-1 polypeptide (e.g., a sNRG-1 polypeptide). Examples of NRG-1 polypeptides that can be detected in a sample obtained from a mammal having prostate cancer include, without limitation, those set forth in the National Center for Biotechnology Information (NCBI) databases at, for example, accession no. XM_024447143.1 (version 109.20210514), accession no. XM_017013368.2 (version 109.20210514), accession no. XP_011542815 (version NP_001309135.1).

Any appropriate method can be used to detect the presence or absence of an elevated level of a NRG-1 polypeptide (e.g., a sNRG-1 polypeptide) within a sample obtained from a mammal (e.g., a human). In some cases, a level of polypeptide expression within a sample can be determined by detecting the presence, absence, or level of the polypeptide in the sample. For example, immunoassays (e.g., immunohistochemistry (IHC) techniques and western blotting techniques), mass spectrometry techniques (e.g., proteomics-based mass spectrometry assays or targeted quantification-based mass spectrometry assays), enzyme-linked immunosorbent assays (ELISAs), radio-immunoassays, and flow cytometry can be used to determine the presence, absence, or level of a polypeptide in a sample. In some cases, a level of polypeptide expression within a sample can be determined by detecting the presence, absence, or level of mRNA encoding the polypeptide in the sample. For example, polymerase chain reaction (PCR)-based techniques such as quantitative RT-PCR techniques, gene expression panel (e.g., next generation sequencing (NGS) such as RNA-seq), and in situ hybridization can be used to determine the presence, absence, or level of mRNA encoding the polypeptide in the sample. In some cases, the presence or absence of an elevated level of a NRG-1 polypeptide (e.g., a sNRG-1 polypeptide) within a sample from a mammal having prostate cancer can be determined as described in Example 1.

When assessing and/or treating a mammal (e.g., a human) having prostate cancer as described herein, the prostate cancer can be any type of prostate cancer. A prostate cancer can be any stage of prostate cancer (e.g., stage I, stage II, stage III, or stage IV). A prostate cancer can be any grade of prostate cancer (e.g., grade 1, grade 2, or grade 3). A prostate cancer can have any Gleason score. In some cases, a prostate cancer can be a primary cancer (e.g., a localized primary cancer). In some cases, a prostate cancer can be a metastatic cancer. In some cases, a prostate cancer can be CSPC. In some cases, a prostate cancer can be CRPC.

In some cases, the methods described herein can include identifying a mammal (e.g., a human) as having prostate cancer. Any appropriate method can be used to identify a mammal as having prostate cancer. For example, physical examination (e.g., a digital rectal examination (DRE)), laboratory testing (e.g., blood tests for prostate-specific antigen (PSA) test), imaging techniques (e.g., ultrasound, magnetic resonance imaging (MRI), bone scan, computerized tomography (CT) scan, and positron emission tomography (PET) scan), and biopsy techniques can be used to identify a mammal (e.g., a human) as having prostate cancer.

In some cases, the methods and materials provided herein can be used to identify a mammal (e.g., a human) as having a metastatic prostate cancer. For example, the presence or absence of an elevated level of a NRG-1 polypeptide in a sample (e.g., a blood sample) obtained from a mammal (e.g., a human) having prostate cancer can be used to determine whether a prostate cancer has metastasized. In some cases, a mammal (e.g., a human) having prostate cancer can be identified as having a metastatic CRPC based, at least in part, on the presence of an elevated level of a NRG-1 polypeptide (e.g., a sNRG-1 polypeptide) in a sample obtained from the mammal.

In some cases, the methods and materials provided herein can be used to identify a mammal (e.g., a human) as having a treatment-resistant prostate cancer e.g., a prostate cancer that it has become resistant or refractory to one or more cancer treatments). For example, the presence or absence of an elevated level of a NRG-1 polypeptide in a sample (e.g., a blood sample) obtained from a mammal (e.g., a human) having prostate cancer can be used to determine whether a prostate cancer has progressed from a CSPC to a treatment-resistant prostate cancer (e.g., a prostate cancer that it has become resistant or refractory to one or more cancer treatments). In some cases, a mammal (e.g., a human) having prostate cancer can be identified as having a treatment-resistant prostate cancer based, at least in part, on the presence of an elevated level of a NRG-1 polypeptide (e.g., a sNRG-1 polypeptide) in a sample obtained from the mammal.

In some cases, the presence or absence of an elevated level of a NRG-1 polypeptide (e.g., a sNRG-1 polypeptide) within a sample from a mammal (e.g., a human) having prostate cancer (e.g., metastatic prostate cancer) can be used to predict survival of the mammal. In some cases, a mammal (e.g., a human) having CSPC (e.g., metastatic CSPC) can be identified as being likely to experience superior survival (e.g., overall survival (OS) and distant progression-free survival (DPFS)) based, at least in part, on the presence of an elevated level of a NRG-1 polypeptide (e.g., a sNRG-1 polypeptide) in a sample obtained from the mammal (e.g., as compared to a mammal having CSPC (e.g., metastatic CSPC) that lacks the presence of an elevated level of a NRG-1 polypeptide such as a sNRG-1 polypeptide). For example, a mammal (e.g., a human) having metastatic CSPC and having a presence of an elevated level of a NRG-1 polypeptide (e.g., a sNRG-1 polypeptide) in a sample obtained from the mammal can be identified as being likely to survive for from about 12 months to about 30 years (e.g., from about 12 months to about 25 years, from about 12 months to about 20 years, from about 12 months to about 15 years, from about 12 months to about 10 years, from about 12 months to about 8 years, from about 12 months to about 5 years, from about 12 months to about 3 years, from about 3 years to about 30 years, from about 5 years to about 30 years, from about 7 years to about 30 years, from about 10 years to about 30 years, from about 15 years to about 30 years, from about 20 years to about 30 years, from about 25 years to about 30 years, from about 2 years to about 25 years, from about 5 years to about 20 years, from about 8 years to about 15 years, from about 10 years to about 12 years, from about 2 years to about 5 years, from about 5 years to about 10 years, from about 10 years to about 15 years, from about 15 years to about 20 years, or from about 20 years to about 25 years). In some cases, a mammal (e.g., a human) having CRPC (e.g., metastatic CRPC) can be identified as being likely to experience inferior survival (e.g., local progression-free survival (LPFS), DPFS, and biochemical progression-free survival (BPFS)) based, at least in part, on the presence of an elevated level of a NRG-1 polypeptide (e.g., a sNRG-1 polypeptide) in a sample obtained from the mammal (e.g., as compared to a mammal having CRPC (e.g., metastatic CRPC) that lacks the presence of an elevated level of a NRG-1 polypeptide such as a sNRG-1 polypeptide). For example, a mammal (e.g., a human) having metastatic CRPC and having a presence of an elevated level of a NRG-1 polypeptide (e.g., a sNRG-1 polypeptide) in a sample obtained from the mammal can be identified as being likely to survive for from about 6 months to about 5 years (e.g., from about 6 months to about 4 years, from about 6 months to about 3 years, from about 6 months to about 2 years, from about 6 months to about 1 year, from about 1 year to about 5 years, from about 2 years to about 5 years, from about 3 years to about 5 years, from about 4 years to about 5 years, from about 1 year to about 4 years, from about 2 years to about 3 years, from about 1 year to about 3 years, or from about 2 years to about 4 years).

When treating a mammal (e.g., a human) having prostate cancer (e.g., CRPC), the mammal can be subjected to one or more (e.g., one, two, three, four, five, or more) therapies that can sensitize the prostate cancer to one or more cancer treatments (e.g., one or more anti-androgen agents). In some cases, a therapy that can sensitize a prostate cancer (e.g., CRPC) to one or more anti-androgen agents can be a therapy that reduces the level of a NRG-1 polypeptide (e.g., a sNRG-1 polypeptide) within the mammal. Examples of therapies that can sensitize a prostate cancer (e.g., CRPC) to one or more anti-androgen agents include, without limitation, radiation therapies (e.g., metastases-directed SBRTs), therapeutic plasma exchange (TPE), and surgeries.

In some cases, when a mammal (e.g., a human) having prostate cancer (e.g., CRPC) is subjected to one or more (e.g., one, two, three, four, five, or more) therapies that can sensitize prostate cancer to one or more cancer treatments (e.g., one or more anti-androgen agents), the mammal also can be administered or instructed to self-administer one or more (e.g., one, two, three, four, five, or more) anti-androgen agents. Examples of anti-androgen agents include, without limitation, leuprolide (e.g., LUPRON DEPOT® and ELIGARD®), goserelin (e.g., ZOLADEX®), triptorelin (e.g., TRELSTAR®), histrelin (e.g., VANTAS®), degarelix (e.g., FIRMAGON®), abiraterone (e.g., ZYTIGA®), ketoconazole (e.g., NIZORAL®), flutamide (e.g., EULEXIN®), bicalutamide (e.g., CASODEX®), nilutamide (e.g., NILANDRON®), enzalutamide (e.g., XTANDI®), apalutamide (e.g., ERLEADA®), darolutamide (e.g., NUBEQA®), and bicalutamide (e.g., CASODEX®). In cases where one or more anti-androgen agents are used together with one or more therapies that can reduce the level of a NRG-1 polypeptide (e.g., a sNRG-1 polypeptide) within the mammal, the one or more therapies that can reduce the level of a NRG-1 polypeptide (e.g., a sNRG-1 polypeptide) within the mammal can be performed at the same time or independently of the administration of the one or more anti-androgen agents. For example, the one or more anti-androgen agents can be administered before, during, or after the one or more therapies that can reduce the level of a NRG-1 polypeptide (e.g., a sNRG-1 polypeptide) within the mammal are performed.

When treating a mammal (e.g., a human) having prostate cancer (e.g., CRPC), the mammal can be administered or instructed to self-administer one or more (e.g., one, two, three, four, five, or more) agents that can sensitize the prostate cancer to one or more cancer treatments (e.g., one or more anti-androgen agents). In some cases, an agent that can sensitize a prostate cancer (e.g., CRPC) to one or more anti-androgen agents can be an inhibitor of NRG-1 polypeptide expression and/or activity. For example, an agent that can sensitize a prostate cancer (e.g., a CRPC) to one or more anti-androgens can be an agent that reduces the level of a NRG-1 polypeptide (e.g., a sNRG-1 polypeptide) within the mammal. In some cases, an agent that can sensitize a prostate cancer (e.g., CRPC) to one or more anti-androgen agents can alter a NRG-1 polypeptide (e.g., a sNRG-1 polypeptide) signaling pathway. Examples of agents that can sensitize a prostate cancer (e.g., CRPC) to one or more anti-androgen agents include, without limitation, inhibitors of a NRG-1 polypeptide, inhibitors of a disintegrin and metalloproteinase domain-containing protein 10 (ADAM10) polypeptide, inhibitors of an ADAM17 polypeptide, inhibitors of a poly (ADP-ribose) polymerase (PARP) polypeptide, agents that can inhibit heterodimerization of a human epidermal growth factor receptor (HER) 2 polypeptide and a HER3 polypeptide (e.g., HER2/HER3 heterodimerization), and NRG-1 neutralizing antibodies.

In some cases, a mammal (e.g., a human) having prostate cancer (e.g., CRPC) can be administered or instructed to self-administer one or more (e.g., one, two, three, four, five, or more) inhibitors of a NRG-1 polypeptide (e.g., to sensitize the prostate cancer to one or more cancer treatments such as one or more anti-androgen agents). In some cases, the one or more inhibitors of a NRG-1 polypeptide can be effective to reduce the level (e.g., the systemic level) of a NRG-1 polypeptide (e.g., a sNRG-1 polypeptide) within a mammal. For example, an inhibitor of a NRG-1 polypeptide can inhibit release of sNRG-1 polypeptide into circulating blood within a mammal. In some cases, an inhibitor of a NRG-1 polypeptide that can inhibit release of sNRG-1 polypeptide into circulating blood within a mammal can reduce or eliminate cleavage of transmembrane NRG-1 polypeptides such that the release of a sNRG-1 polypeptide in circulating blood within a mammal is reduced or eliminated. An inhibitor of a NRG-1 polypeptide can be an inhibitor of NRG-1 polypeptide activity (e.g., anti-NRG-1 antibodies such as neutralizing anti-NRG-1 antibodies and small molecules that target a NRG-1 polypeptide) or an inhibitor of NRG-1 polypeptide expression (e.g., nucleic acid molecules designed to induce RNA interference of NRG-1 polypeptide expression such as siRNA molecules and shRNA molecules). Examples of inhibitors of a NRG-1 polypeptide include, without limitation, 8a4 (e.g., Creative Biolabs, TAB-583MZ), 10b2M3 (e.g., Creative Biolabs, NRP-0422-P1898), 10bM3 (e.g., Creative Labs, TAB-584MZ-F(E)), rucaparib, olaparib, sorafenib, and TAPI-2. In some cases, an inhibitor of a NRG-1 polypeptide can be as described elsewhere (see, e.g., Zhang et al., Cancer Cell, 38(2):279-296.e9 (2020)).

In some cases, a mammal (e.g., a human) having prostate cancer (e.g., CRPC) can be administered or instructed to self-administer one or more (e.g., one, two, three, four, five, or more) inhibitors of an ADAM10 polypeptide (e.g., to sensitize the prostate cancer to one or more cancer treatments such as one or more anti-androgen agents). In some cases, the one or more inhibitors of a NRG-1 polypeptide can be effective to reduce a systemic level of NRG-1 polypeptides (e.g., sNRG-1 polypeptides) within a mammal. An inhibitor of an ADAM10 polypeptide can be an inhibitor of ADAM10 polypeptide activity (e.g., anti-ADAM10 antibodies such as anti-ADAM10 antibodies that can target the catalytic domain of an ADAM10 polypeptide and small molecules that target an ADAM10 polypeptide) or an inhibitor of ADAM10 polypeptide expression (e.g., nucleic acid molecules designed to induce RNA interference of ADAM10 polypeptide expression such as siRNA molecules and shRNA molecules). Examples of inhibitors of an ADAM10 polypeptide include, without limitation, INCB8765, GI 254023X, TAPI-0, and TAPI-2. In some cases, an inhibitor of an ADAM10 polypeptide also can be an inhibitor of an ADAM17 polypeptide. Examples of inhibitors of ADAM10/ADAM17 polypeptides include, without limitation, TAPI-0, TAPI-2, and D1A antibody. In some cases, an inhibitor of an ADAM10 polypeptide can be as described elsewhere (see, e.g., Smith, Jr. et al., Front. Immunol., 11:499 (2020)).

In some cases, a mammal (e.g., a human) having prostate cancer (e.g., CRPC) can be administered or instructed to self-administer one or more (e.g., one, two, three, four, five, or more) inhibitors of an ADAM17 polypeptide (e.g., to sensitize the prostate cancer to one or more cancer treatments such as one or more anti-androgen agents). In some cases, the one or more inhibitors of a NRG-1 polypeptide can be effective to reduce a systemic level of NRG-1 polypeptides (e.g., sNRG-1 polypeptides) within a mammal. An inhibitor of an ADAM17 polypeptide can be an inhibitor of ADAM17 polypeptide activity (e.g., anti-ADAM17 antibodies such as neutralizing anti-ADAM17 antibodies and small molecules that target an ADAM17 polypeptide) or an inhibitor of ADAM17 polypeptide expression (e.g., nucleic acid molecules designed to induce RNA interference of ADAM17 polypeptide expression such as siRNA molecules and shRNA molecules). Examples of inhibitors of an ADAM17 polypeptide include, without limitation, anti-ADAM17 D1(A12), TAPI-0, and TAPI-2. In some cases, an inhibitor of an ADAM17 polypeptide also can be an inhibitor of an ADAM10 polypeptide. Examples of inhibitors of ADAM10/ADAM17 polypeptides include, without limitation, TAPI-0, TAPI-2, and D1A antibody. In some cases, an inhibitor of an ADAM17 polypeptide can be as described elsewhere (see, e.g., Ni et al., Medicine (Kaunas), 56(7):322 (2020)).

In some cases, a mammal (e.g., a human) having prostate cancer (e.g., CRPC) can be administered or instructed to self-administer one or more (e.g., one, two, three, four, five, or more) inhibitors of a PARP polypeptide (e.g., to sensitize the prostate cancer to one or more cancer treatments such as one or more anti-androgen agents). In some cases, the one or more inhibitors of a NRG-1 polypeptide can be effective to reduce a systemic level of NRG-1 polypeptides (e.g., sNRG-1 polypeptides) within a mammal. An inhibitor of a PARP polypeptide can be an inhibitor of PARP polypeptide activity (e.g., anti-PARP antibodies such as neutralizing anti-PARP antibodies and small molecules that target a PARP polypeptide) or an inhibitor of PARP polypeptide expression (e.g., nucleic acid molecules designed to induce RNA interference of PARP polypeptide expression such as siRNA molecules and shRNA molecules). Examples of inhibitors of a PARP polypeptide include, without limitation, olaparib, rucaparib, niraparib, and talazoparib. In some cases, an inhibitor of a PARP polypeptide can be as described elsewhere (see, e.g., Adashek et al., Cells, 8(8):860 (2019); Geethakumari et al., Curr. Treat. Options Oncol., 18(6):37 (2017); and de Bono et al., N. Engl. J. Med., 382(22):2091-2102 (2020)).

In some cases, a mammal (e.g., a human) having prostate cancer (e.g., CRPC) can be administered or instructed to self-administer one or more (e.g., one, two, three, four, five, or more) agents that can inhibit HER2/HER3 heterodimerization (e.g., to sensitize the prostate cancer to one or more cancer treatments such as one or more anti-androgen agents). In some cases, the one or more agents that can inhibit HER2/HER3 heterodimerization can be effective to alter a NRG-1 polypeptide (e.g., a sNRG-1 polypeptide) signaling pathway. In some cases, the one or more agents that can inhibit HER2/HER3 heterodimerization can induce homodimerization of two HER3 polypeptides (e.g., HER3 homodimerization). Examples of agents that can inhibit HER2/HER3 heterodimerization include, without limitation, trastuzumab (e.g., HERCEPTIN®), ARRY-380, erlotinib, gefitinib, afatinib, neratinib, and pertuzumab (e.g., PERJETA®). In some cases, an inhibitor of HER2/HER3 heterodimerization can be as described elsewhere (see, e.g., Claus et al., eLife 7:e32271 (2018); Morris et al., Cancer, 94:980-986 (2002); and De Bono et al., Artic. J. Clin. Oncol., 25:257-262 (2007)).

In some cases when treating a mammal (e.g., a human) having prostate cancer (e.g., CRPC), the mammal is not administered any agent that can induce HER2/HER3 heterodimerization. For example, a mammal having CRPC and treated as described herein can be treated in a manner that avoids administering lapatinib (e.g., TYVERB/TYKERB®). For example, a mammal having CRPC and treated as described herein can be treated in a manner that avoids administering an agent that can induce HER2/HER3 heterodimerization that is as described elsewhere (see, e.g., Whang et al., Urol. Oncol., 31:82-6 (2013)).

In some cases, when a mammal (e.g., a human) having prostate cancer (e.g., CRPC) is administered one or more (e.g., one, two, three, four, five, or more) agents that can sensitize prostate cancer to one or more cancer treatments (e.g., one or more anti-androgen agents), the mammal also can be administered or instructed to self-administer one or more (e.g., one, two, three, four, five, or more) anti-androgen agents. Examples of anti-androgen agents include, without limitation, leuprolide (e.g., LUPRON DEPOT® and ELIGARD®), goserelin (e.g., ZOLADEX®), triptorelin (e.g., TRELSTAR®), histrelin (e.g., VANTAS®), degarelix (e.g., FIRMAGON®), abiraterone (e.g., ZYTIGA®), ketoconazole (e.g., NIZORAL®), flutamide (e.g., EULEXIN®), bicalutamide (e.g., CASODEX®), nilutamide (e.g., NILANDRON®), enzalutamide (e.g., XTANDI®), apalutamide (e.g., ERLEADA®), darolutamide (e.g., NUBEQA®), and bicalutamide (e.g. CASODEX). In some cases, the one or more anti-androgen agents can be administered together with the one or more agents that can sensitize a prostate cancer to one or more anti-androgen agents (e.g., can be administered together in a single composition). In some cases, the one or more anti-androgen agents can be administered independent of the one or more agents that can sensitize a prostate cancer to one or more anti-androgen agents. When the one or more anti-androgen agents are administered independent of the one or more agents that can sensitize a prostate cancer to one or more anti-androgen agents, the one or more agents that can sensitize a prostate cancer to one or more anti-androgen agents can be administered first, and the one or more anti-androgen agents administered second, or vice versa.

In some cases, when treating a mammal (e.g., a human) having prostate cancer as described herein, the treatment can be effective to treat the cancer. For example, the number of cancer cells present within a mammal can be reduced using the methods and materials described herein. In some cases, the size (e.g., volume) of one or more tumors present within a mammal can be reduced using the methods and materials described herein. For example, the methods and materials described herein can be used to reduce the size of one or more tumors present within a mammal having prostate cancer by, for example, 10, 20, 30, 40, 50, 60, 70, 80, 90, 95, or more percent. In some cases, the size (e.g., volume) of one or more tumors present within a mammal does not increase.

In some cases, when treating a mammal (e.g., a human) having prostate cancer as described herein, the treatment can be effective to improve survival of the mammal. For example, the methods and materials described herein can be used to improve disease-free survival (e.g., relapse-free survival). For example, the methods and materials described herein can be used to improve progression-free survival. For example, the methods and materials described herein can be used to improve the survival of a mammal having prostate cancer by, for example, 10, 20, 30, 40, 50, 60, 70, 80, 90, 95, or more percent. For example, the methods and materials described herein can be used to improve the survival of a mammal having prostate cancer by, for example, at least 6 months (e.g., about 6 months, about 8 months, about 10 months, about 1 year, about 1.5 years, about 2 years, about 2.5 years, or about 3 years).

In some cases, when treating a mammal (e.g., a human) having prostate cancer as described herein, the treatment can be effective to reduce one or more symptoms of the cancer. Examples of symptoms of prostate cancer include, without limitation, trouble urinating, decreased force in the stream of urine, blood in the urine, blood in the semen, bone pain, losing weight without trying, erectile dysfunction, fatigue, anemia, cachexia, and neuropathy. For example, the methods and materials described herein can be used to reduce one or more symptoms within a mammal having prostate cancer by, for example, 10, 20, 30, 40, 50, 60, 70, 80, 90, 95, or more percent.

In some cases, when treating a mammal (e.g., a human) having prostate cancer (e.g., CRPC) as described herein (e.g., by administering one or more agents and/or one or more therapies that can sensitize prostate cancer to one or more anti-androgen agents and, optionally, one or more anti-androgen agents), the one or more agents and/or one or more therapies that can sensitize prostate cancer to one or more cancer treatments (e.g., one or more anti-androgen agents) and, optionally, one or more anti-androgen agents can be the only cancer treatment(s) administered to the mammal.

In some cases, when treating a mammal (e.g., a human) having prostate cancer (e.g., CRPC) as described herein (e.g., by administering one or more agents and/or one or more therapies that can sensitize prostate cancer to one or more anti-androgen agents and, optionally, one or more anti-androgen agents), the mammal also can be treated with one or more additional agents/therapies used to treat cancer. Examples of additional agents/therapies used to treat cancer include, without limitation, surgery, chemotherapies, targeted therapies (e.g., monoclonal antibody therapies), angiogenesis inhibitors, immunomodulators, checkpoint blockade therapies, radiotherapies, bone-directed therapies, and isotope-containing therapies. In cases where one or more agents and/or one or more therapies that can sensitize prostate cancer to one or more cancer treatments (e.g., one or more anti-androgen agents) and, optionally, one or more anti-androgen agents are used in combination with one or more additional agents/therapies, the one or more additional agents/therapies can be administered at the same time or independently. For example, the one or more agents and/or one or more therapies that can sensitize prostate cancer to one or more cancer treatments (e.g., one or more anti-androgen agents) and, optionally, one or more anti-androgen agents can be administered first, and the one or more additional agents/therapies can be administered second, or vice versa.

The invention will be further described in the following examples, which do not limit the scope of the invention described in the claims.

### EXAMPLES

### Example 1: Mesenchymal Stem Cell-Derived Systemic NRG-1 Treatment of Metastatic Prostate Cancer

This example demonstrates that host mesenchymal stem cells (MSCs) release sNRG-1 polypeptides, which can be suppressed by radiotherapy, ADAM inhibitors, or PARP inhibitors to restore CRPC sensitivity to androgen deprivation therapy (ADT) in vivo.

### MATERIALS AND METHODS

### Patient Samples

Patients with oligometastatic castration-sensitive prostate cancer (CSPC) or castration-resistant prostate cancer (CRPC) with three or fewer metastatic PET-11C-choline-positive lesions who were referred for stereotactic body radiation therapy (SBRT) were enrolled prospectively to a single arm study in which patient blood was drawn just prior to and fourteen days after SBRT. Definitions for castration resistance were according to the Prostate Cancer Clinical Trials Working Group (Scher et al., J. Clin. Oncol., 26:1148-59 (2008)). Plasma levels of sNRG-1 were determined by ELISA (R&D Biosystems) according to manufacturer protocols as understood by those skilled in the art. In some instances, MTS assays were performed.

### Results

### NRG-1 is a Systemic Molecular Switch in the Progression of Prostate Cancer.

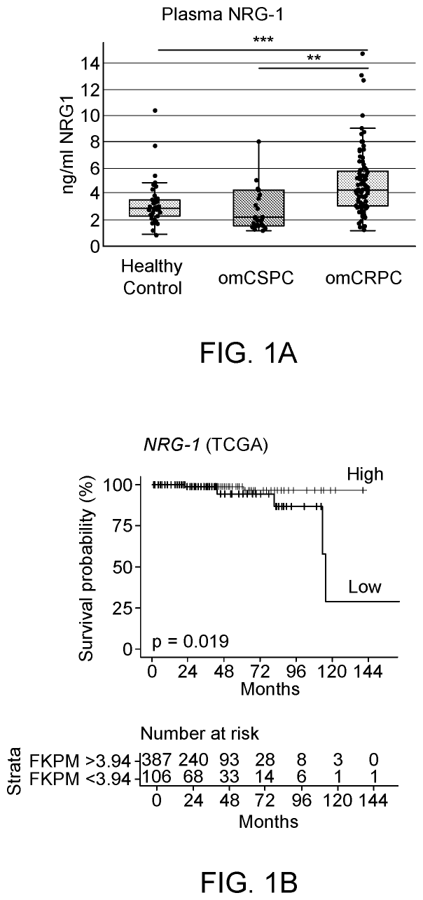

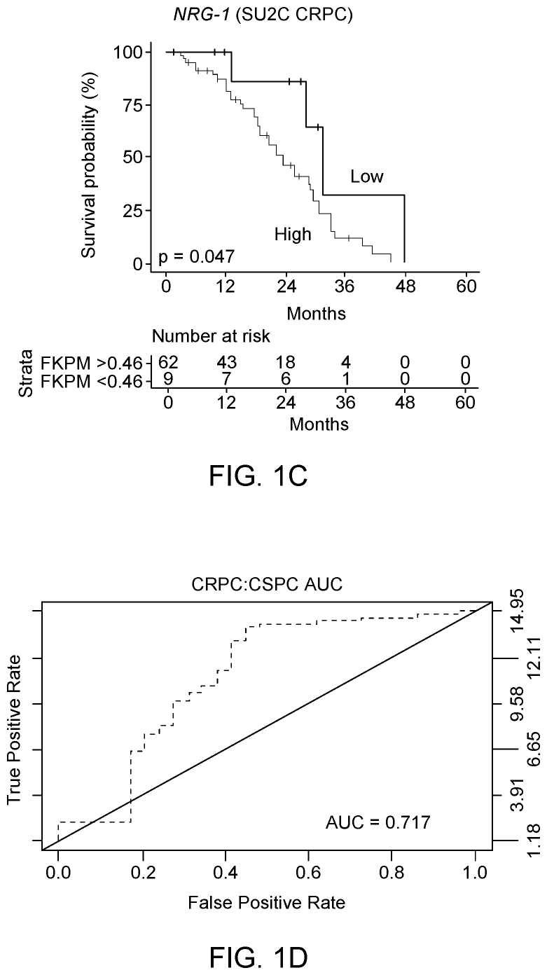

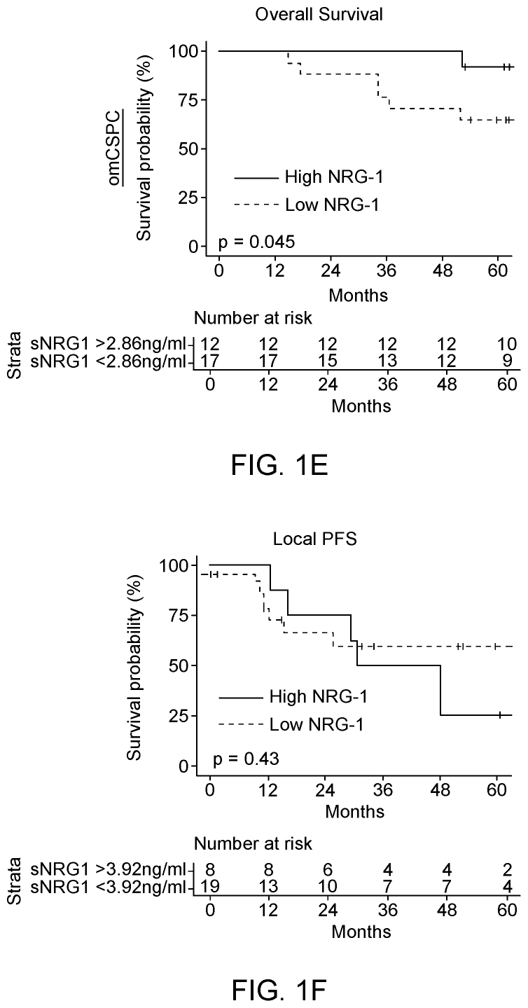

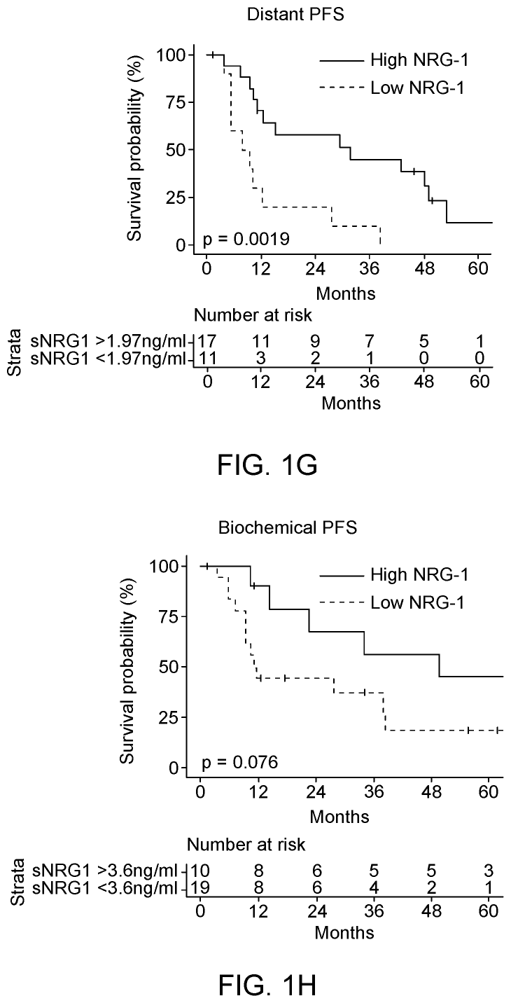

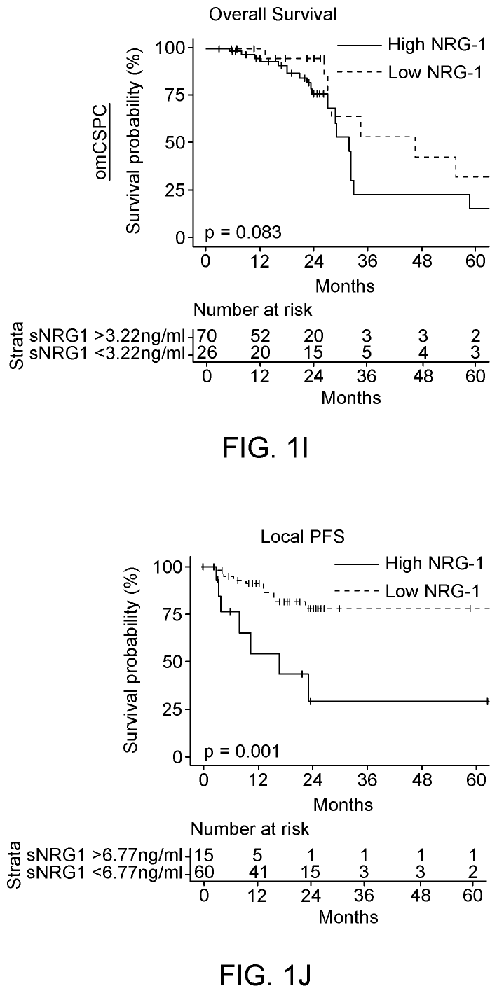

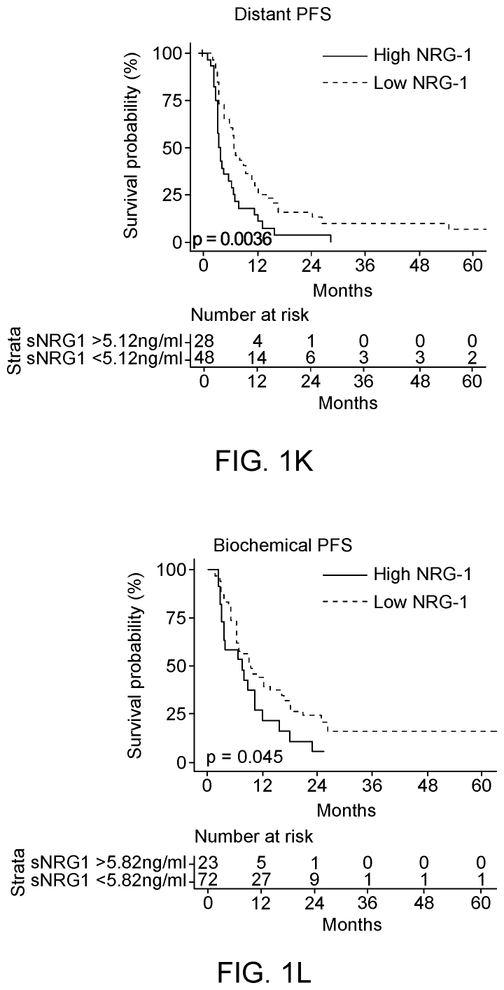

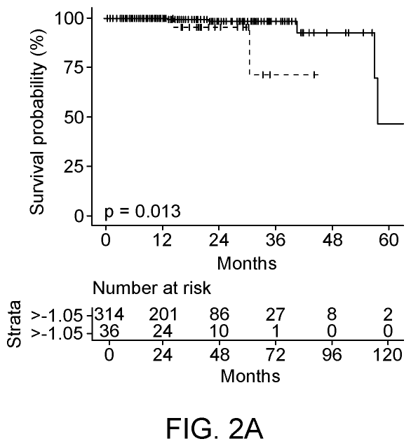

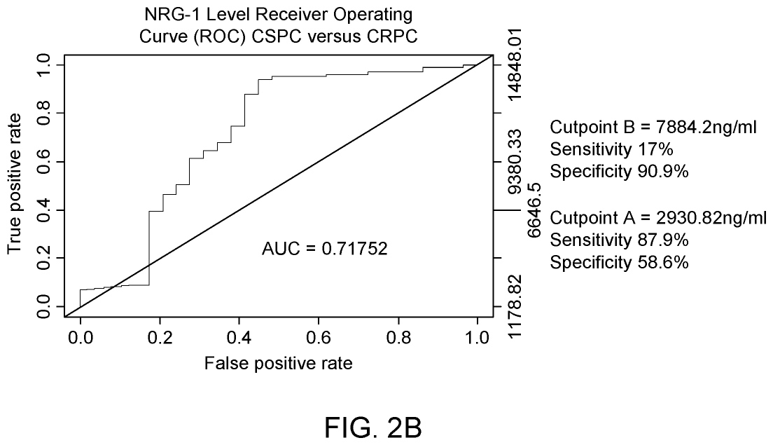

Blood samples from patients with oligometastatic prostate cancer prior to planned stereotactic body radiation therapy (SBRT) and from healthy control subjects were analyzed. Significantly elevated NRG-1 polypeptide levels were detected in patients with oligometastatic CRPC (mean 4.89 ng/mL, 95% CI 4.49-5.28 ng/ml) versus healthy controls (mean 3.2 ng/mL, 95% CI 2.84-3.55 ng/mL, p<0.0001) or patients with oligometastatic CSPC (mean 3.39 ng/ml, 95% CI 2.67-4.12 ng/ml, p<0.0001) (FIG. 1A). To determine whether local tumor NRG-1 polypeptide expression predicts outcomes in prostate cancer, the Cancer Genome Atlas (TCGA) (see, for example, Uhlen, M. et al. Science 357, (2017)) was queried for tumor bed NRG-1 mRNA expression and compared clinical outcomes. Patients with very high NRG-1 mRNA expression in these CSPC tumors experienced improved survival (FIG. 1B) and with NRG-1 polypeptides in the same samples as measured by RPPA (FIGS. 2A-2B). However, high NRG-1 mRNA expression paradoxically predicted poor survival in a dataset consisting entirely of CRPC tumors (FIGS. 1C-1D).

To determine the impact of systemic NRG-1 polypeptides in the cohort of CSPC patients from FIG. 1A, overall survival (OS), local progression-free survival (LPFS), distant progression-free survival (DPFS), and biochemical progression-free survival (BPFS) were analyzed by NRG-1 polypeptide levels (FIGS. 1E-1H). In CSPC patients, high systemic NRG-1 polypeptides predicted generally superior outcomes including OS and DPFS versus low systemic NRG-1 polypeptides. Patients with oligometastatic CRPC showed a strikingly paradoxical trend; high systemic NRG-1 polypeptides predicted inferior LPFS, DPFS, and BPFS (FIGS. 1I-1L). These data suggest that NRG-1 polypeptides may be part of a systemic molecular switch in the progression of prostate cancer.

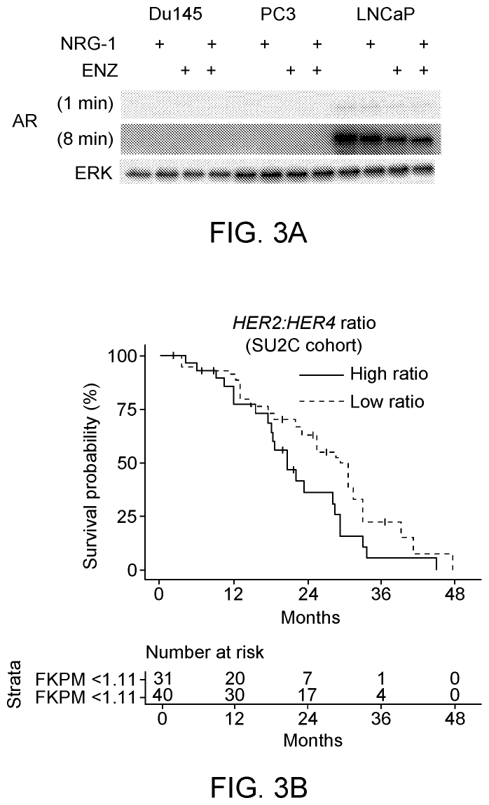

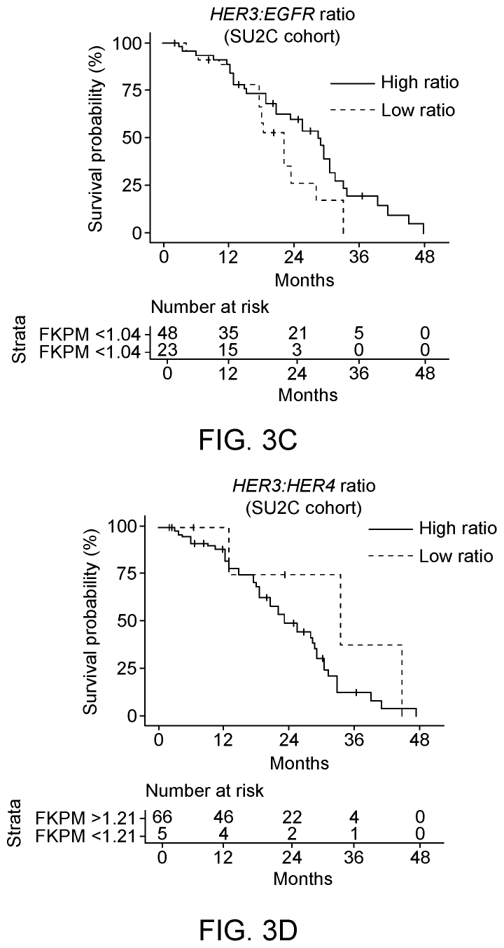

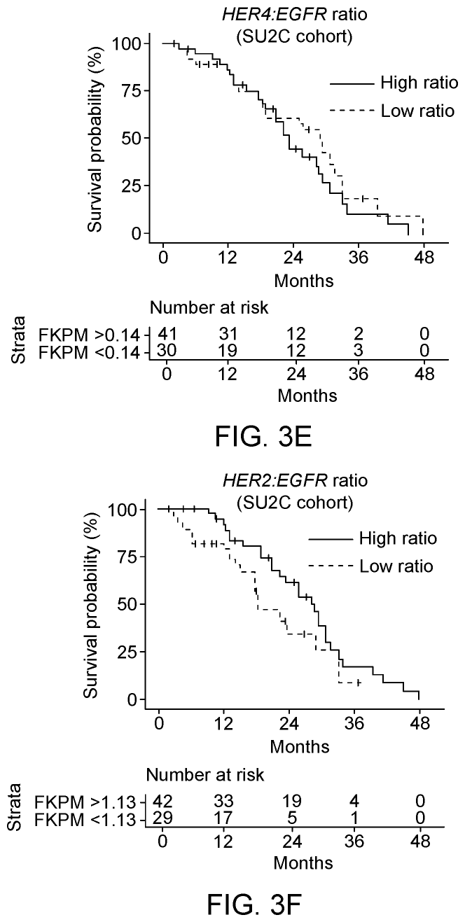

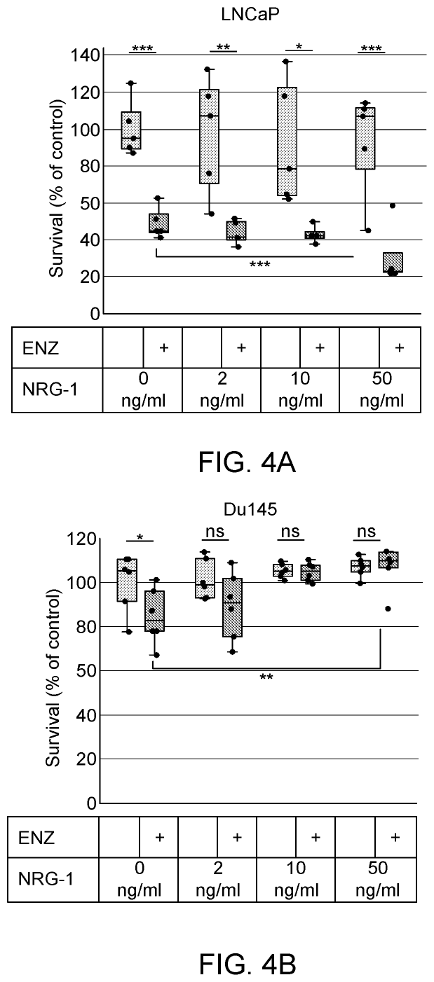

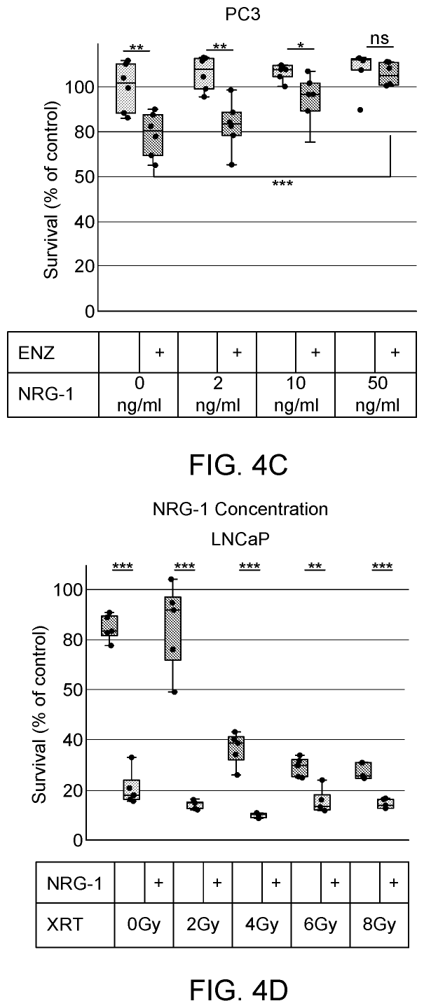

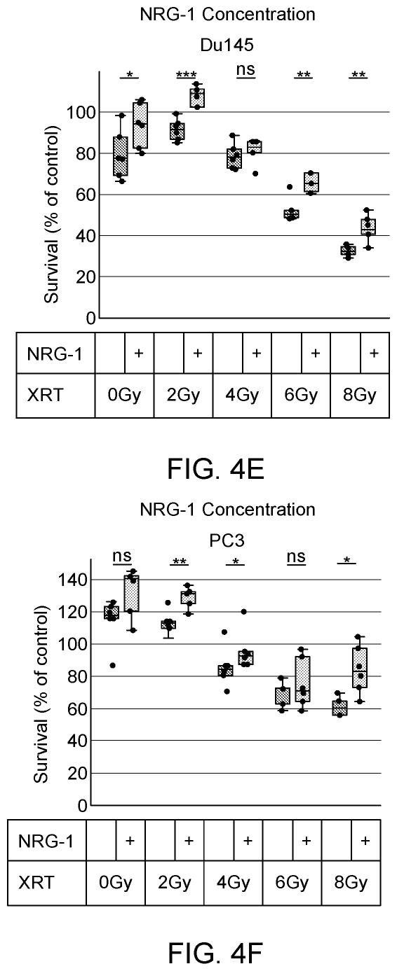

It was next examined whether NRG-1 polypeptides differentially affect antiandrogen-sensitive and resistant prostate cancer cells. Antiandrogen-sensitive LNCaP prostate cancer cells express androgen receptor, whereas antiandrogen-resistant Du145 and PC3 cells express very limited androgen receptor (FIG. 3A). Antiandrogen-sensitive LNCaP and antiandrogen-resistant Du145 and PC3 cells were treated with increasing concentrations of recombinant human NRG-1 polypeptides in the presence of vehicle control versus enzalutamide (Enz) and measured cell survival by a quantitative colony forming assay. Both Enz and NRG-1 polypeptide treatment induced death of LNCaP cells in an additive fashion (FIG. 4A). In contrast, NRG-1 polypeptides rescued Du145 and PC3 cells in a dose-dependent manner from Enz-induced cell death (FIG. 4B).

The paradoxical toxic and salutary effects of NRG-1 polypeptides on antiandrogen-sensitive versus antiandrogen-resistant cells mirrors the outcomes in CSPC and CRPC patients, respectively. Prostate cancer cell lines were treated with vehicle versus NRG-1 polypeptides (50 ng/mL) in the presence of 3 μM enzalutamide with increasing doses of photon radiation. NRG-1 polypeptides and radiotherapy led to the killing of LNCaP cells in an additive manner (FIG. 4C). Conversely, NRG-1 polypeptide treatment consistently improved the survival of radio-resistant Du145 and PC3 cells for each dose of radiation (FIG. 4D).

The opposing cytotoxic and resistance-inducing effects of NRG-1 polypeptides on antiandrogen-sensitive and antiandrogen-resistant prostate cancer cells, respectively, was commensurate with the clinical observation of patients with CSPC and CRPC (Table 1).

TABLE 1
NRG-1 biomarker characteristics.

| | Castration- | Castration- | | Androgen- |
|---|---|---|---|---|
| | sensitive | resistant | Androgen- | resistant |
| | prostate | prostate | sensitive | cells |
| | cancer | cancer | cells | (Du145, |
| | (CSPC) | (CRPC) | (LNCaP) | PC3) |
| High | Superior OS, | Inferior | Tumor cell | Tumor cell |
| NRG-1 | DPFS, BPFS | LPFS, DPFS, | death and | survival and |
| | | BPFS | sensitivity | resistance |
| HER2:HER3 | Low | High | Low | High |
| ratio | | | | |

overall survival (OS), local progression-free survival (LPFS), distant progression-free survival (DPFS), and biochemical progression-free survival (BPFS)

### Prostate Cancer Cells Induce Mesenchymal Stem Cells to Produce NRG-1 in a PARPi- and ADAMs-Dependent Manner

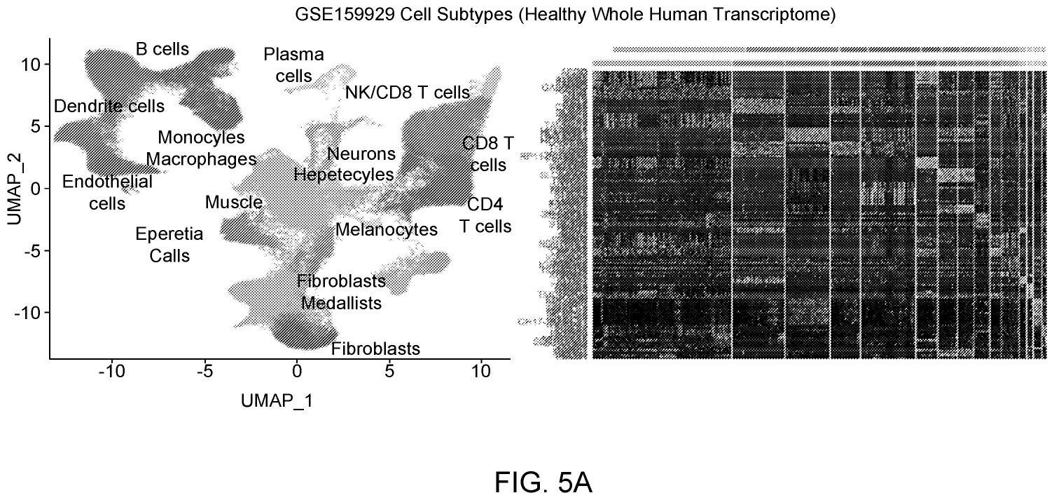

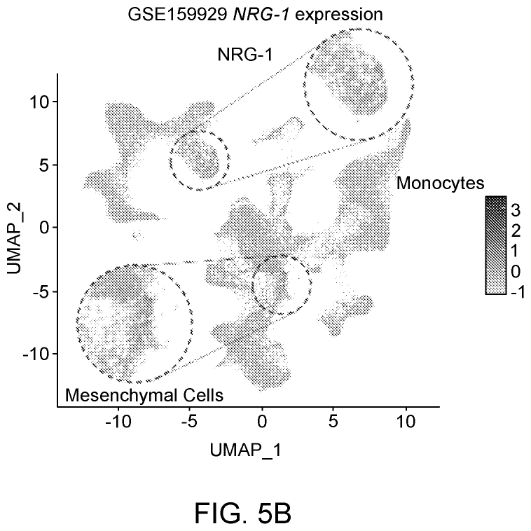

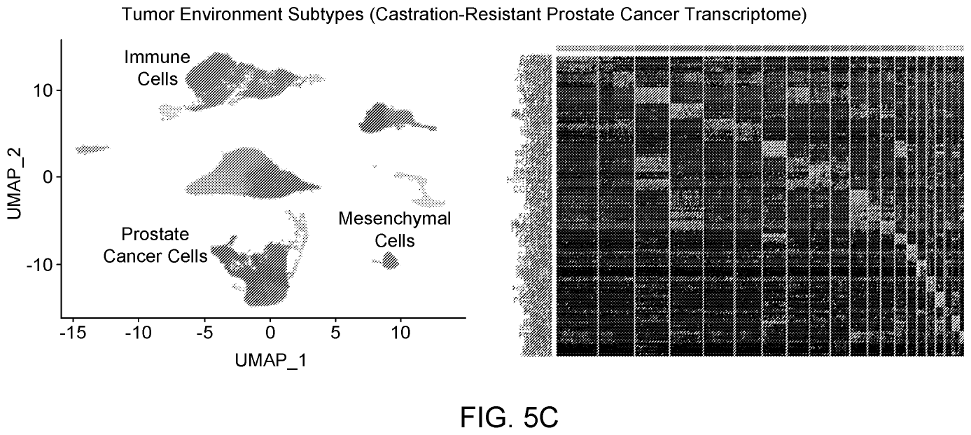

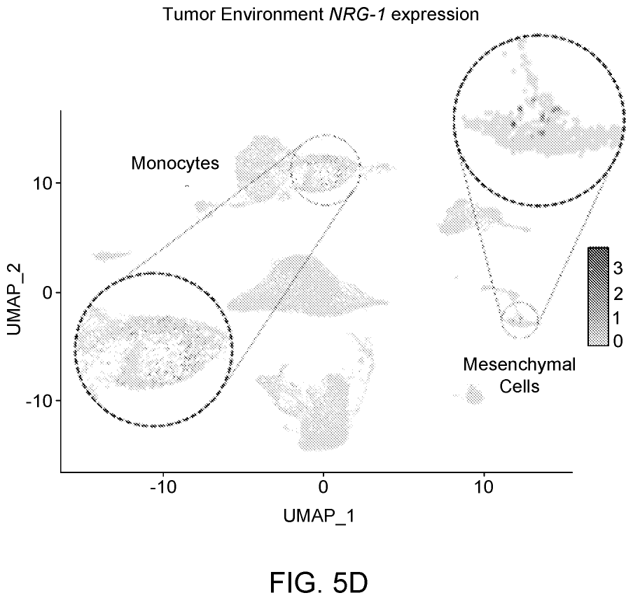

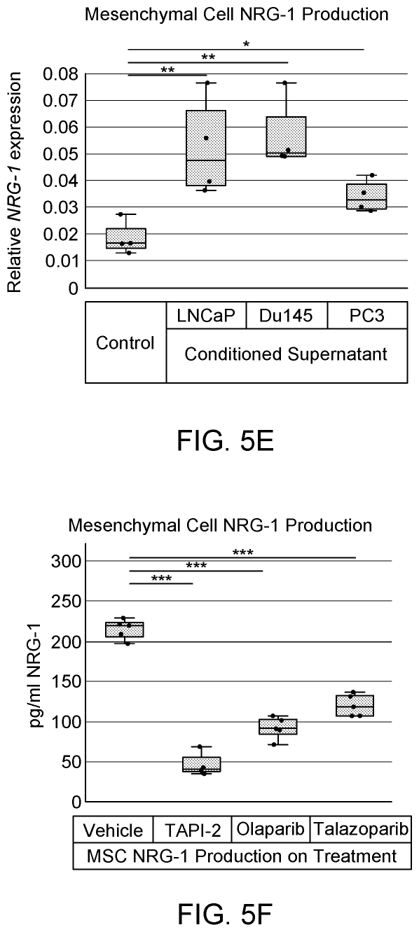

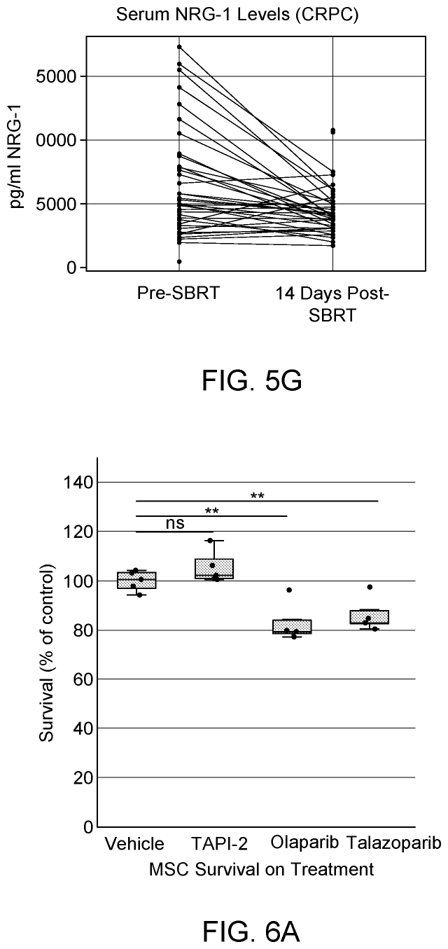

To determine possible sources of NRG-1 polypeptides, a composite single-cell whole human transcriptome was queried for NRG-1 polypeptide expression (FIGS. 5A-5B). Monocytes and stem-like MSCs expressed significant NRG-1 polypeptides. A composite single-cell CRPC tumor microenvironment transcriptome was also queried (FIGS. 5C-5D). In the CRPC tumor microenvironment, MSCs and monocytes produced NRG-1 polypeptides at low levels.

To validate MSC NRG-1 polypeptide production, lines of MSC outgrowth cells were tested for NRG-1 polypeptide production. Bone outgrowth MSCs produce NRG-1 polypeptides, whereas further-differentiated cells in the mesenchymal lineage did not. MSCs were treated with supernatants from prostate cancer cell lines and NRG-1 transcription was measured by RT-PCR (FIG. 5E). Prostate cancer cell supernatants induced elevated NRG-1 transcription.

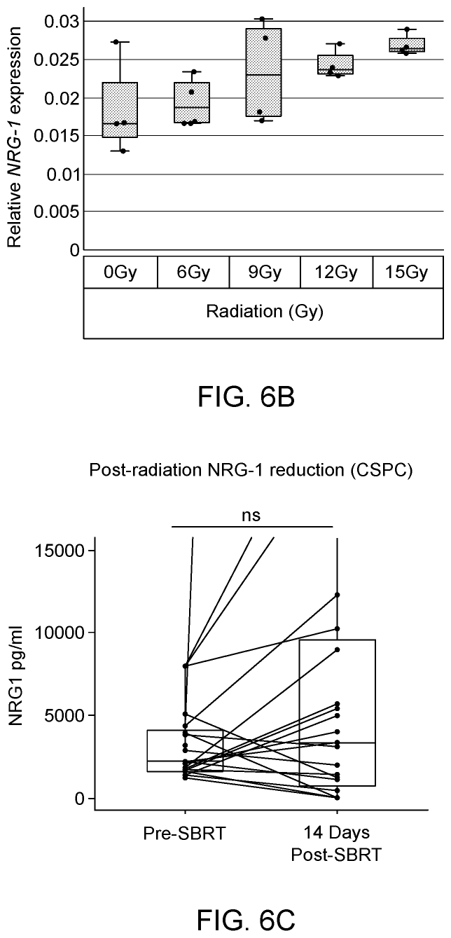

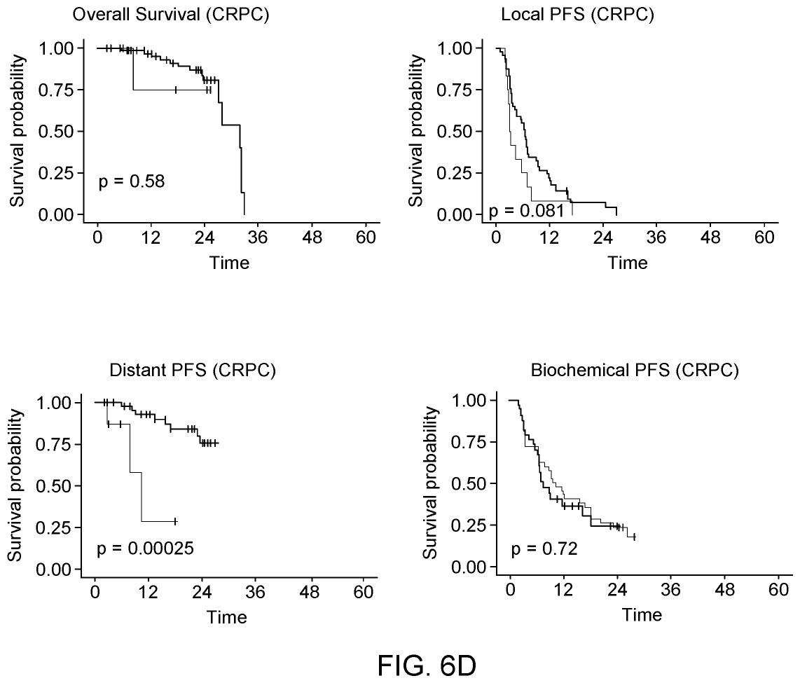

To find methods for reducing NRG-1 polypeptide production in CRPC, tests with an array of inhibitors were performed. A primary human mesenchymal stem cell line that produces NRG-1 polypeptides was treated with an array of inhibitors versus vehicle control. Inhibitors of ADAM10/ADAM17 or Poly (ADP-ribose) polymerase (PARP) reduced production without commensurate decrease in MSC viability (FIGS. 5F and 6A). Radiation did not reduce NRG-1 polypeptide expression by MSCs (FIG. 6B). In patients with oligometastatic CRPC (but not CSPC) who underwent radiotherapy, levels of plasma NRG-1 polypeptides decreased after treatment (FIG. 5G and FIG. 6C), possibly reflecting the ability of prostate cancer cells to induce MSC NRG-1 polypeptide production. For patients with oligometastatic CRPC, remaining day 14 post-radiotherapy plasma NRG-1 polypeptide levels predicted inferior distant progression-free survival (FIG. 6D).

### The NRG-1 Molecular Switch in Prostate Cancer Mediated by Tumor Cell Surface HER2 and HER3

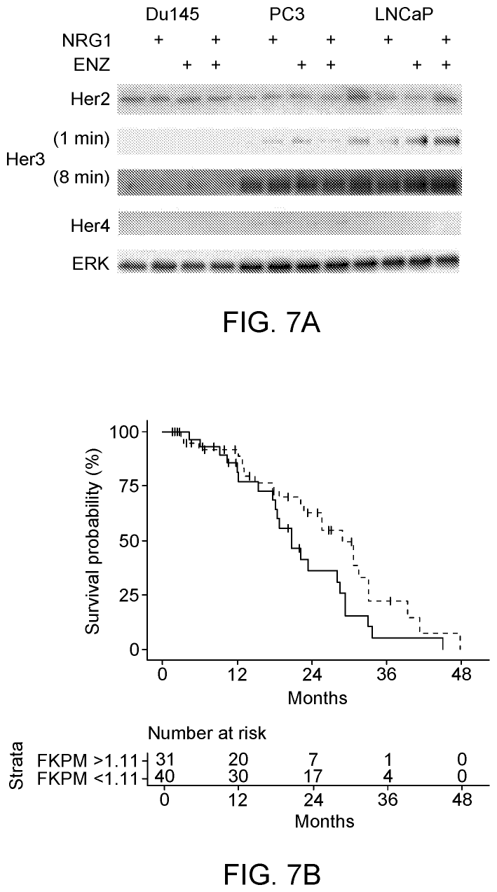

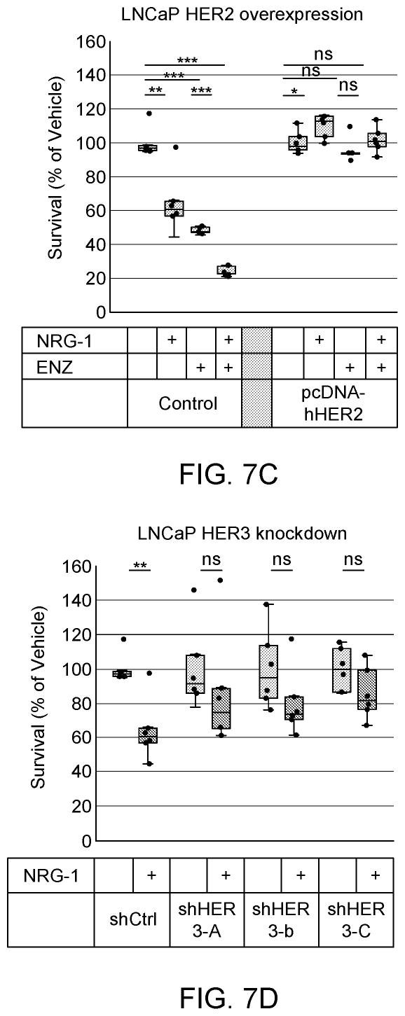

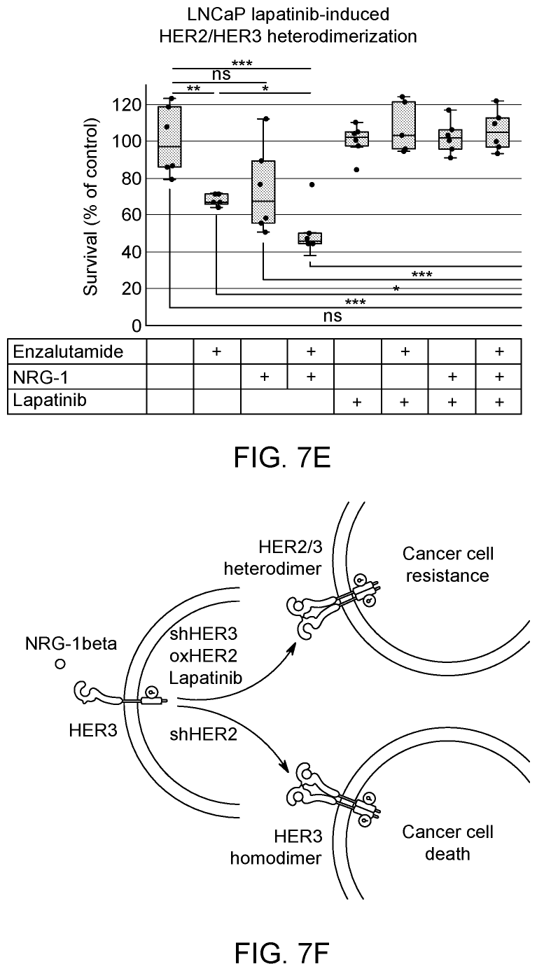

While either castration-sensitive or castration-resistant prostate cells may induce distant mesenchymal stem cells to produce NRG-1 polypeptides, the downstream sequelae of NRG-1 polypeptide signaling diverges in CSPC versus CRPC. It was examined whether different receptor combinations are engaged by NRG-1 polypeptides. On Western blot analysis, LNCaP cells express high levels of HER3 relative to Du145 and PC3 cells, while all cells express varying degrees of HER2; HER4 is not highly expressed in prostate cancer cells (FIG. 7A). Treatment with NRG-1 polypeptides or enzalutamide downregulates AR in LNCaP cells (FIG. 3). In an analysis of EGFR family member expression in a cohort of patients with CRPC, a low HER2 to HER3 ratio trended toward poor overall survival (FIG. 7B). No other ratio of EGFR family members predicted survival in CRPC on analysis (FIGS. 3B-3F).

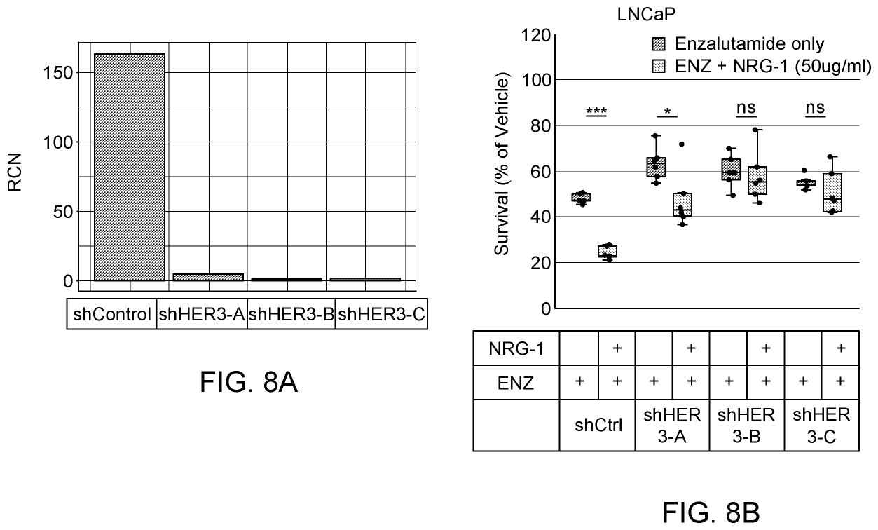

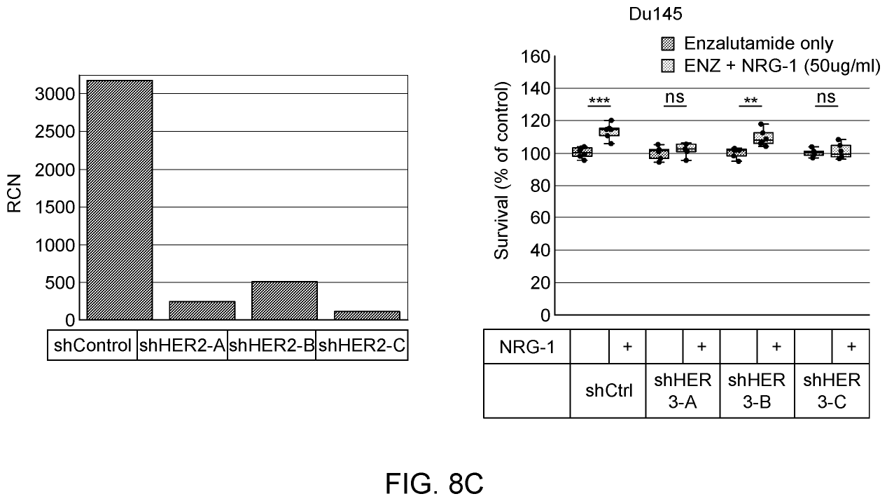

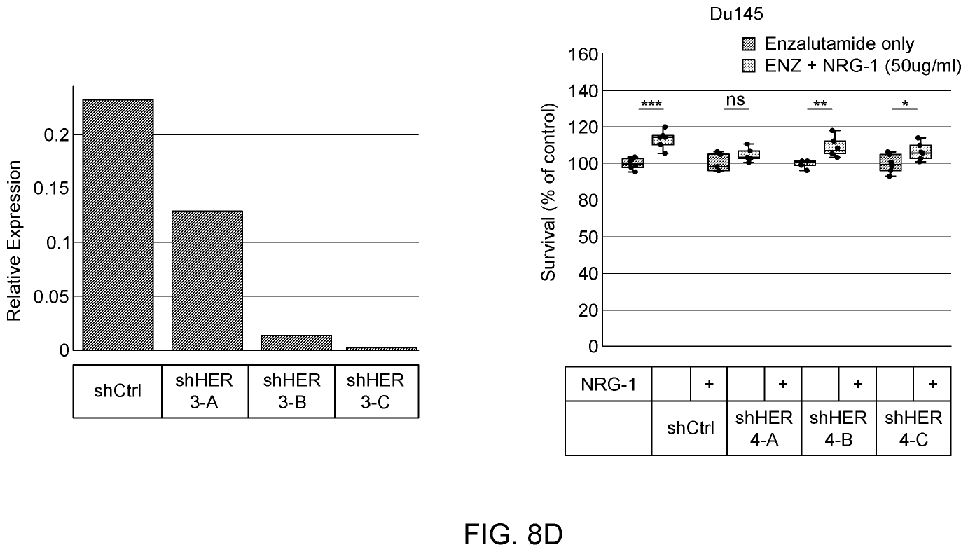

To examine whether HER3 may rescue androgen-sensitive cells from NRG-1-induced killing, LNCaP cells were transfected with an empty vector versus a wild-type HER2 expression vector and treated with vehicle versus NRG-1 polypeptides and/or enzalutamide. The addition of HER2 rescued cells from NRG-1 polypeptide-induced killing and from enzalutamide (FIG. 7C). LNCaP cells were then transfected with control shRNA versus shRNA targeting HER3 (FIG. 8A). Knockdown of HER3 rescued LNCaP cells from NRG-1- and enzalutamide-induced cell death (FIG. 7D and FIG. 8B). Relatedly, knockdown of HER2—but not HER4—reduces or eliminates Du145 cell benefit from NRG-1 polypeptide treatment (FIG. 8C-8D). To further confirm that HER2/HER3 heterodimerization can mediate a pro-survival response to NRG-1 polypeptides in CRPC, LNCaP cells were treated with lapatinib that induces HER2/HER3 heterodimerization. Pre-treatment of cells with lapatinib abolished NRG-1- and antiandrogen-mediated cell death (FIG. 7E).

Together, these observations suggest that HER3 homodimerization leads to prostate cancer cell death, whereas heterodimerization of HER3 with HER2 leads to prostate cancer cell survival and antiandrogen and radiotherapy resistance (FIG. 5F). Consequently, HER2 and HER3 receptor availability determine the molecular switch of NRG-1 polypeptides in prostate cancer.

### Example 2: Sensitizing CRPC to Androgen Treatment

A biological sample (e.g., a blood sample such as plasma) is obtained from a human having CRPC. The obtained sample is examined for the presence or absence of an elevated level of NRG-1 polypeptides. If the presence of an elevated level of NRG-1 polypeptides is detected in the sample, then the human is subjected to metastases-directed SBRT and/or TPE to lower the level of NRG-1 polypeptides within the human. The metastases-directed SBRT and/or TPE is/are effective to sensitize the CRPC to androgen treatment.

### Example 3: Treating CRPC

A biological sample (e.g., a blood sample such as plasma) is obtained from a human having CRPC. The obtained sample is examined for the presence or absence of an elevated level of NRG-1 polypeptides. If the presence of an elevated level of NRG-1 polypeptides is detected in the sample, then the human is subjected to metastases-directed SBRT and/or TPE to lower the level of NRG-1 polypeptides within the human and sensitize the CRPC to androgen treatment, and is administered one or more anti-androgen agents. The administered anti-androgen agents can reduce number of cancer cells within the human.

### Example 4: Sensitizing CRPC to Androgen Treatment

A biological sample (e.g., a blood sample such as plasma) is obtained from a human having CRPC. The obtained sample is examined for the presence or absence of an elevated level of NRG-1 polypeptides. If the presence of an elevated level of NRG-1 polypeptides is detected in the sample, then the human is administered one or more inhibitors of a NRG-1 polypeptide (e.g., rucaparib, olaparib, sorafenib, and TAPI-2), one or more inhibitors of an ADAM10 polypeptide (e.g., INCB8765, GI 254023X, TAPI-0, and TAPI-2), one or more inhibitors of an ADAM17 polypeptide (e.g., anti-ADAM17 D1(A12), TAPI-0, and TAPI-2), and/or one or more inhibitors of a PARP polypeptide (e.g., olaparib, rucaparib, niraparib, and talazoparib) to lower the level of NRG-1 polypeptides within the human. The one or more inhibitors of a NRG-1 polypeptide is/are effective to sensitize the CRPC to androgen treatment.

### Example 5: Treating CRPC

A biological sample (e.g., a blood sample such as plasma) is obtained from a human having CRPC. The obtained sample is examined for the presence or absence of an elevated level of NRG-1 polypeptides. If the presence of an elevated level of NRG-1 polypeptides is detected in the sample, then the human is administered one or more inhibitors of a NRG-1 polypeptide (e.g., rucaparib, olaparib, sorafenib, and TAPI-2), one or more inhibitors of an ADAM10 polypeptide (e.g., INCB8765, GI 254023X, TAPI-0, and TAPI-2), one or more inhibitors of an ADAM17 polypeptide (e.g., anti-ADAM17 D1(A12), TAPI-0, and TAPI-2), and/or inhibitors of a PARP polypeptide (e.g., olaparib, rucaparib, niraparib, and talazoparib) to lower the level of NRG-1 polypeptides within the human and sensitize the CRPC to androgen treatment, and is administered one or more anti-androgen agents. The administered anti-androgen agents can reduce number of cancer cells within the human.

### Example 6: Sensitizing CRPC to Androgen Treatment

A biological sample (e.g., a blood sample such as plasma) is obtained from a human having CRPC. The obtained sample is examined for the presence or absence of an elevated level of NRG-1 polypeptides. If the presence of an elevated level of NRG-1 polypeptides is detected in the sample, then the human is administered one or more agents that can inhibit HER2/HER3 heterodimerization (e.g., trastuzumab, ARRY-380, erlotinib, gefitinib, afatinib, neratinib, and pertuzumab) to alter NRG-1 polypeptide signaling within the human. The one or more agents that can inhibit HER2/HER3 heterodimerization is/are effective to sensitize the CRPC to androgen treatment.

### Example 7: Treating CRPC

A biological sample (e.g., a blood sample such as plasma) is obtained from a human having CRPC. The obtained sample is examined for the presence or absence of an elevated level of NRG-1 polypeptides. If the presence of an elevated level of NRG-1 polypeptides is detected in the sample, then the human is administered one or more agents that can inhibit HER2/HER3 heterodimerization (e.g., trastuzumab, ARRY-380, erlotinib, gefitinib, afatinib, neratinib, and pertuzumab) to sensitize the CRPC to androgen treatment, and is administered one or more anti-androgen agents. The administered anti-androgen agents can reduce number of cancer cells within the human.

### Example 8: PARP Inhibition Reduces Systemic NRG-1 Levels

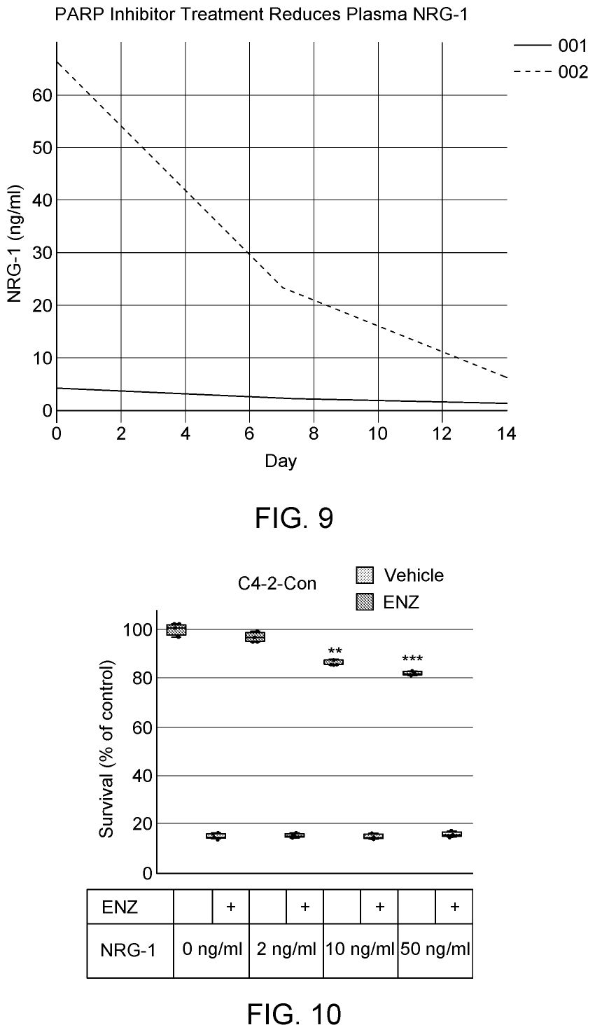

Blood levels NRG-1 of two patients with cancer were measured prior to and during treatment with niraparib or olaparib. Prior to treatment, patients had rising levels of NRG-1. Blood levels of NRG-1 were reduced over the course of treatment (FIG. 9). Notably, the patient receiving niraparib experienced a rising NRG-1 level just prior to disease relapse, suggesting NRG-1 as a biomarker of recurrence in this cancer.

### Example 9: Sensitizing CRPC to Androgen Treatment

The results in this Example represent and expand on at least some of the results provided in other Examples.

To further determine the effects of NRG-1 on prostate cancer cells, including cells that are susceptible to 2nd generation anti-androgen agents, C4-2-Con cells were treated with increasing concentrations of NRG-1 as well as enzalutamide (ENZ) (FIG. 10). Cell survival was measured by MTS assay. Increasing concentrations of NRG-1 led to increased cell this sensitive cell type.

C4-2-Con cells were grown in DMEM with 5% FBS, 10 μM HEPES, and Pen/Strep at 200 k/mL in 96-well plates and seeded for colony forming units (CFU). Treatments with increasing concentrations of NRG-1 were performed overnight after which media were refreshed for 48 hours. Colony-forming units were visualized after fixation in 50% TCA at 4 degrees followed by SRB assay to measure cell survival.

### Example 10: NRG-1 and Survival in CRPC Cells

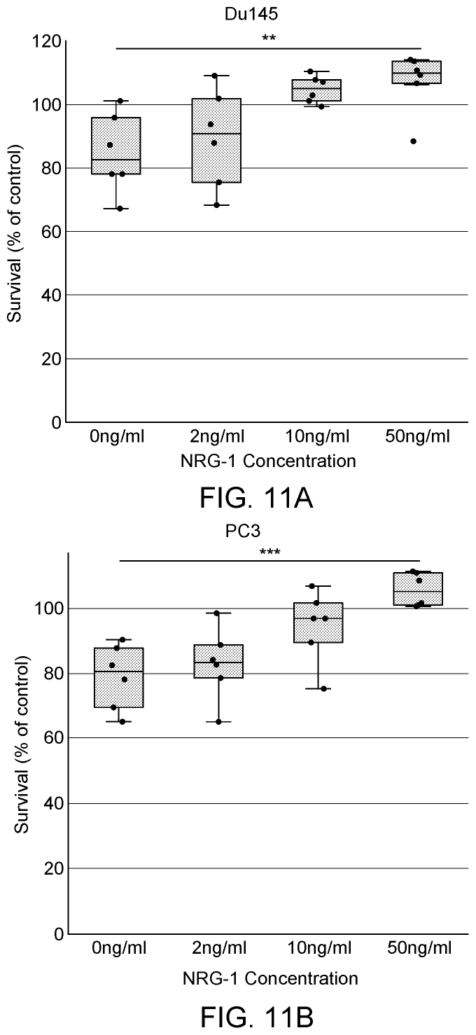

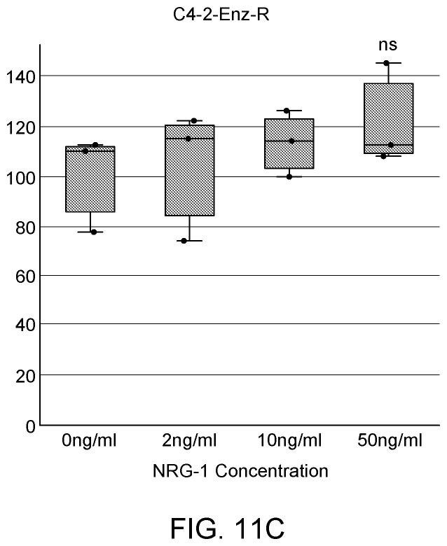

Highly resistant cell lines DU145, PC3, and C4-2-Enz-R were treated with increasing concentrations of NRG-1. The results showed that increasing concentrations of NRG-1 led to paradoxically increased cell survival in these cell lines in a dose-dependent manner (FIG. 11).

NRG-1 increased survival in CRPC cells (FIG. 11)

DU145, PC3, and C4-2-Enz-R cells were individually grown in either RPMI or DMEM with 5% FBS, 10 μM HEPES, and Pen/Strep at 200 k/mL in 96-well plates and seeded for colony forming units (CFU). Treatments increasing concentrations of NRG-1 were performed overnight after which media were refreshed for 48 hours. Colony-forming units were visualized after fixation in 50% TCA at 4 degrees followed by SRB assay to measure cell survival.

### Example 11: NRG-1 in Prostate Cancer

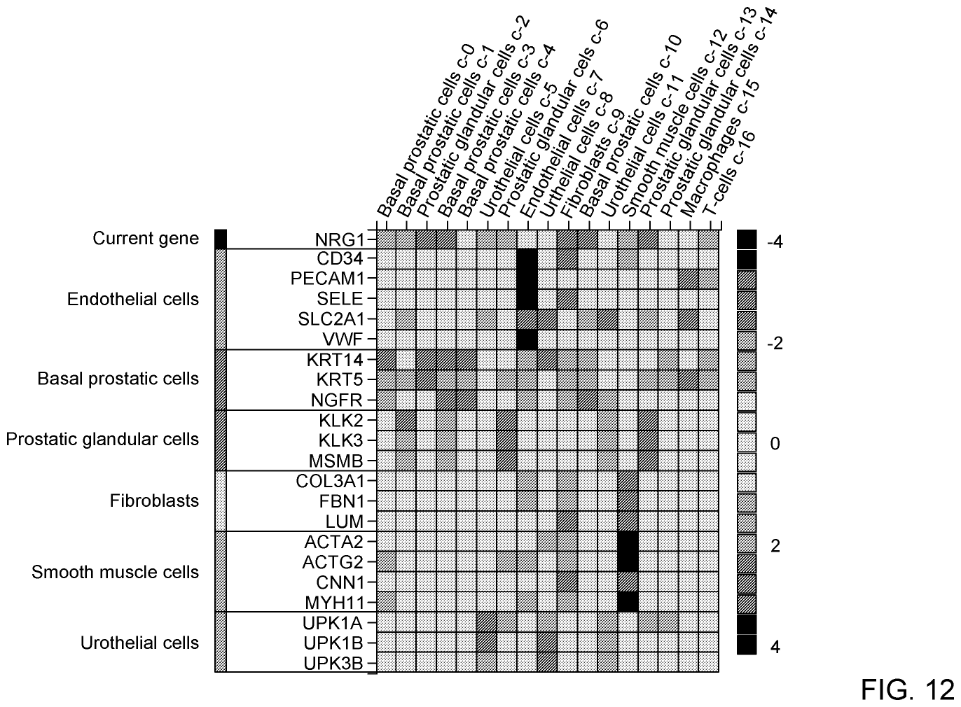

Publicly available single cell RNAseq data of the prostate cancer environment were queried and it was found that mesenchymal cells were the clear source of this NRG-1 in vivo. This was performed with multiple data sets accessible from GEO and other reports. NRG-1 was produced by mesenchymal cells in patients with prostate cancer (FIG. 12).

### Example 12. PARP Inhibitors Reduce Systemic NRG-1

Blood levels of NRG-1 of five patients with cancer were measured prior to and during treatment with PARP inhibitors. Prior to treatment, patients had rising levels of NRG-1.

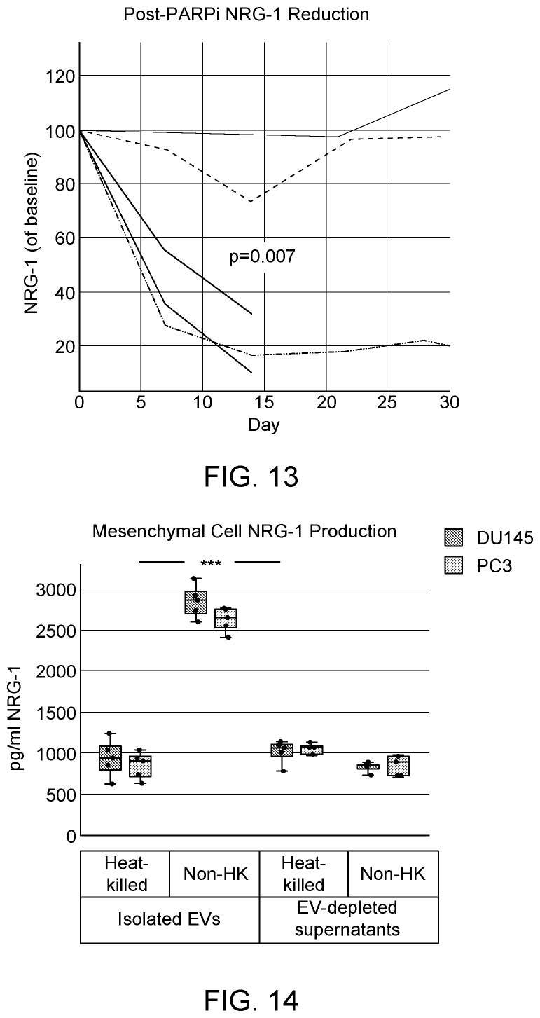

PARP inhibitors significantly reduce systemic NRG-1 in patients treated with these compounds (FIG. 13). These results demonstrate that PARP inhibition can reduce systemic NRG-1 in mesenchymal cells and can combat NRG-1-mediated resistance (e.g., NRG-1-mediated resistance to ADT, antiandrogens, chemotherapy, and/or radiation therapy).

### Example 13. Prostate Cancer Cell-Derived Extracellular Vesicles (P-EVs) Induce Mesenchymal Cell NRG-1 Production

A soluble factor from prostate cancer cells induced the production of NRG-1 by mesenchymal cells. Extracellular vesicles (EVs) were isolated from prostate cancer cell lines DU145 and PC3 by 100 kDa cutoff column by ultracentrifugation and measured by flow cytometry and by direct visualization microscopy. Mesenchymal cells were treated with isolated EVs, heat-killed EVs, or EV-depleted supernatants.

Isolated P-EVs, but not heat-killed EVs or EV-depleted supernatants, induced mesenchymal cell NRG-1 production (FIG. 14). These results demonstrate that methods that remove or block EVs (e.g., TPE) can resensitize prostate cancers to treatment.

### Example 14. Patient-Derived Xenografts (PDX)

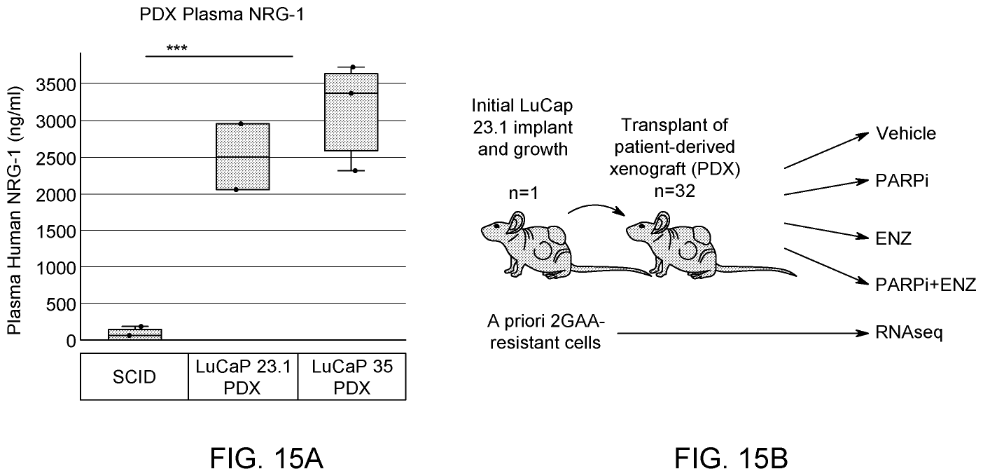

The plasma concentrations of NRG-1 were measured from background SCID mice, patient derived xenograft (PDX) models LuCaP 23.1 and LuCaP 35. Mouse blood was collected by cardiac puncture on sacrifice. Plasma was separated by ultracentrifugation at room temperature for 5 minutes. ELISA was performed using R&D NRG-1beta DuoSet ELISA kit. PDXs showed detectable systemic NRG-1 (FIG. 15A).

A study design for evaluating systemic NRG-1 in vivo using PDX experiments is shown in FIG. 15B. Treatment with PARP inhibitor or ADAM10/ADAM17 inhibitor improves response to one or more second-generation antiandrogens (e.g., enzalutamide (ENZ).

### Example 15: Exemplary Embodiments

Embodiment 1. A method for assessing a mammal having prostate cancer, wherein said method comprises:
(a) detecting an elevated level of a NRG-1 polypeptide in a blood sample from said mammal;
(b) classifying said mammal as having a CRPC if said presence of said elevated level is detected; and
(c) classifying said mammal as not having said CRPC if said absence of said elevated level is detected.

Embodiment 2. The method of embodiment 1, wherein said mammal is a human.

Embodiment 3. The method of any one of embodiments 1-2, wherein said blood sample is plasma.

Embodiment 4. The method of any one of embodiments 1-3, wherein said elevated comprises at least 3 nanograms of said NRG-1 polypeptide per milliliter of said blood sample (ng/ml).

Embodiment 5. The method of any one of embodiments 1-4, wherein said method comprises detecting said presence of said elevated level of said polypeptide.

Embodiment 6. The method of embodiment 5, wherein said method comprises classifying said mammal as having said CRPC prostate cancer.

Embodiment 7. The method of any one of embodiments 1-4, wherein said method comprises detecting said absence of said elevated level of said polypeptide.

Embodiment 8. The method of embodiment 7, wherein said method comprises classifying said mammal as not having said CRPC prostate cancer.

Embodiment 9. The method of any one of embodiments 1-8, wherein said prostate cancer is a metastatic prostate cancer.

Embodiment 10. A method for restoring androgen sensitivity to a CRPC within a mammal, wherein said method comprises subjecting said mammal to a therapy that reduces a systemic level of a NRG-1 polypeptide within said mammal.

Embodiment 11. The method of embodiment 10, wherein said mammal is a human.

Embodiment 12. The method of embodiments 10 or embodiment 11, wherein said therapy is selected from the group consisting of metastases-directed SBRT and TPE.

Embodiment 13. A method for restoring androgen sensitivity to a CRPC within a mammal, wherein said method comprises administering an inhibitor of a NRG-1 polypeptide to said mammal.

Embodiment 14. The method of embodiment 13, wherein said mammal is a human.

Embodiment 15. The method of any one of embodiments 13-14, wherein said method is effective to reduce a systemic level of a NRG-1 polypeptide within said mammal.

Embodiment 16. The method of any one of embodiments 13-15, wherein said inhibitor of said NRG-1 polypeptide is an inhibitor of NRG-1 polypeptide activity.

Embodiment 17. The method of any one of embodiments 13-15, wherein said inhibitor of said NRG-1 polypeptide is an inhibitor of NRG-1 polypeptide expression.

Embodiment 18. The method of any one of embodiments 13-15, wherein said inhibitor of said NRG-1 polypeptide is selected from the group consisting of rucaparib, olaparib, sorafenib, and TAPI-2.

Embodiment 19. The method of any one of embodiments 10-18, wherein said CRPC is a metastatic CRPC.

Embodiment 20. A method for restoring androgen sensitivity to a CRPC within a mammal, wherein said method comprises administering an inhibitor of ADAM10 polypeptide to said mammal.

Embodiment 21. The method of embodiment 20, wherein said mammal is a human.

Embodiment 22. The method of any one of embodiments 20-21, wherein said method is effective to reduce a systemic level of a NRG-1 polypeptide within said mammal.

Embodiment 23. The method of any one of embodiments 20-21, wherein said inhibitor of said ADAM10 polypeptide is an inhibitor of ADAM10 polypeptide activity.

Embodiment 24. The method of any one of embodiments 20-21, wherein said inhibitor of said ADAM10 polypeptide is an inhibitor of ADAM10 polypeptide expression.

Embodiment 25. The method of any one of embodiments 20-21, wherein said inhibitor of said NRG-1 polypeptide is selected from the group consisting of INCB8765, GI 254023X, TAPI-0, and TAPI-2.

Embodiment 26. The method of any one of embodiments 20-25, wherein said CRPC is a metastatic CRPC.

Embodiment 27. A method for restoring androgen sensitivity to a CRPC within a mammal, wherein said method comprises administering an inhibitor of an ADAM17 polypeptide to said mammal.

Embodiment 28. The method of embodiment 27, wherein said mammal is a human.

Embodiment 29. The method of any one of embodiments 27-28, wherein said method is effective to reduce a systemic level of a NRG-1 polypeptide within said mammal.

Embodiment 30. The method of any one of embodiments 27-28, wherein said inhibitor of said ADAM17 polypeptide is an inhibitor of ADAM17 polypeptide activity.

Embodiment 31. The method of any one of embodiments 27-28, wherein said inhibitor of said ADAM17 polypeptide is an inhibitor of ADAM17 polypeptide expression.

Embodiment 32. The method of any one of embodiments 27-28, wherein said inhibitor of said NRG-1 polypeptide is selected from the group consisting of an anti-ADAM17 D1(A12) antibody, TAPI-0, and TAPI-2.

Embodiment 33. The method of any one of embodiments 27-32, wherein said CRPC is a metastatic CRPC.

Embodiment 34. A method for restoring androgen sensitivity to a CRPC within a mammal, wherein said method comprises administering an inhibitor of a PARP polypeptide to said mammal.

Embodiment 35. The method of embodiment 34, wherein said mammal is a human.

Embodiment 36. The method of any one of embodiments 34-35, wherein said method is effective to reduce a systemic level of a NRG-1 polypeptide within said mammal.

Embodiment 37. The method of any one of embodiments 34-35, wherein said inhibitor of said PARP polypeptide is an inhibitor of PARP polypeptide activity.

Embodiment 38. The method of any one of embodiments 34-35, wherein said inhibitor of said PARP polypeptide is an inhibitor of PARP polypeptide expression.

Embodiment 39. The method of any one of embodiments 34-35, wherein said inhibitor of said NRG-1 polypeptide is selected from the group consisting of olaparib, rucaparib, niraparib, and talazoparib.

Embodiment 40. The method of any one of embodiments 34-39, wherein said CRPC is a metastatic CRPC.

Embodiment 41. A method for restoring androgen sensitivity to a CRPC within a mammal, wherein said method comprises administering an agent that can inhibit HER2/HER3 heterodimerization within said mammal.

Embodiment 42. The method of embodiment 41, wherein said mammal is a human.

Embodiment 43. The method of any one of embodiments 41-42, wherein said agent can induce HER3 homodimerization.

Embodiment 44. The method of any one of embodiments 41-42, wherein said agent is selected from the group consisting of trastuzumab, ARRY-380, erlotinib, gefitinib, afatinib, neratinib, and pertuzumab.

Embodiment 45. The method of any one of embodiments 41-44, wherein said CRPC is a metastatic CRPC.

Embodiment 46. A method for treating a mammal having CRPC, wherein said method comprises:
(a) subjecting said mammal to a therapy that can reduce a systemic level of NRG-1 polypeptides within said mammal; and
(b) administering an anti-androgen agent to said mammal.

Embodiment 47. The method of embodiment 46, wherein said mammal is a human.

Embodiment 48. The method of any one of embodiments 46-47, wherein said therapy is selected from the group consisting of metastases-directed SBRT and TPE.

Embodiment 49. The method of any one of embodiments 46-48, wherein said anti-androgen agent is selected from the group consisting of leuprolide, goserelin, triptorelin, histrelin, degarelix, abiraterone, ketoconazole, flutamide, bicalutamide, nilutamide, enzalutamide, apalutamide, darolutamide, and bicalutamide.

Embodiment 50. The method of any one of embodiments 46-49, wherein said CRPC is a metastatic CRPC.

Embodiment 51. A method for treating a mammal having CRPC, wherein said method comprises:
(a) administering an inhibitor of a NRG-1 polypeptide to said mammal; and
(b) administering an anti-androgen agent to said mammal.

Embodiment 52. The method of embodiment 51, wherein said mammal is a human.

Embodiment 53. The method of any one of embodiments 51-52, wherein said inhibitor of said NRG-1 polypeptide is an inhibitor of NRG-1 polypeptide activity.

Embodiment 54. The method of any one of embodiments 51-52, wherein said inhibitor of said NRG-1 polypeptide is an inhibitor of NRG-1 polypeptide expression.

Embodiment 55. The method of any one of embodiments 51-52, wherein said inhibitor of said NRG-1 polypeptide is selected from the group consisting of rucaparib, olaparib, sorafenib, and TAPI-2.

Embodiment 56. The method of any one of embodiments 51-55, wherein said CRPC is a metastatic CRPC.

Embodiment 57. The method of any one of embodiments 51-56, wherein said anti-androgen agent is selected from the group consisting of leuprolide, goserelin, triptorelin, histrelin, degarelix, abiraterone, ketoconazole, flutamide, bicalutamide, nilutamide, enzalutamide, apalutamide, darolutamide, and bicalutamide.

Embodiment 58. A method for treating a mammal having CRPC, wherein said method comprises:
(a) administering an inhibitor of an ADAM10 polypeptide to said mammal; and
(b) administering an anti-androgen agent to said mammal.

Embodiment 59. The method of embodiment 58, wherein said mammal is a human.

Embodiment 60. The method of any one of embodiments 58-59, wherein said inhibitor of said ADAM10 polypeptide is an inhibitor of ADAM10 polypeptide activity.

Embodiment 61. The method of any one of embodiments 58-59, wherein said inhibitor of said ADAM10 polypeptide is an inhibitor of ADAM10 polypeptide expression.

Embodiment 62. The method of any one of embodiments 58-59, wherein said inhibitor of said ADAM10 polypeptide is selected from the group consisting of INCB8765, GI 254023X, TAPI-0, and TAPI-2.

Embodiment 63. The method of any one of embodiments 58-62, wherein said CRPC is a metastatic CRPC.

Embodiment 64. The method of any one of embodiments 51-63, wherein said anti-androgen agent is selected from the group consisting of leuprolide, goserelin, triptorelin, histrelin, degarelix, abiraterone, ketoconazole, flutamide, bicalutamide, nilutamide, enzalutamide, apalutamide, darolutamide, and bicalutamide.

Embodiment 65. A method for treating a mammal having CRPC, wherein said method comprises:
(a) administering an inhibitor of an ADAM17 polypeptide to said mammal; and
(b) administering an anti-androgen agent to said mammal.

Embodiment 66. The method of embodiment 65, wherein said mammal is a human.

Embodiment 67. The method of any one of embodiments 65-66, wherein said inhibitor of said ADAM17 polypeptide is an inhibitor of ADAM17 polypeptide activity.

Embodiment 68. The method of any one of embodiments 65-66, wherein said inhibitor of said ADAM17 polypeptide is an inhibitor of ADAM17 polypeptide expression.

Embodiment 69. The method of any one of embodiments 65-66, wherein said inhibitor of said ADAM17 polypeptide is selected from the group consisting of an anti-ADAM17 D1(A12) antibody, TAPI-0, and TAPI-2.

Embodiment 70. The method of any one of embodiments 65-69, wherein said CRPC is a metastatic CRPC.

Embodiment 71. The method of any one of embodiments 65-70, wherein said anti-androgen agent is selected from the group consisting of leuprolide, goserelin, triptorelin, histrelin, degarelix, abiraterone, ketoconazole, flutamide, bicalutamide, nilutamide, enzalutamide, apalutamide, darolutamide, and bicalutamide.

Embodiment 72. A method for treating a mammal having CRPC, wherein said method comprises:
(a) administering an inhibitor of a PARP polypeptide to said mammal; and
(b) administering an anti-androgen agent to said mammal.

Embodiment 73. The method of embodiment 72, wherein said mammal is a human.

Embodiment 74. The method of any one of embodiments 72-73, wherein said inhibitor of said PARP polypeptide is an inhibitor of PARP polypeptide activity.

Embodiment 75. The method of any one of embodiments 72-73, wherein said inhibitor of said PARP polypeptide is an inhibitor of PARP polypeptide expression.

Embodiment 76. The method of any one of embodiments 72-73, wherein said inhibitor of said PARP polypeptide is selected from the group consisting of olaparib, rucaparib, niraparib, and talazoparib.

Embodiment 77. The method of any one of embodiments 72-76, wherein said CRPC is a metastatic CRPC.

Embodiment 78. The method of any one of embodiments 72-77, wherein said anti-androgen agent is selected from the group consisting of leuprolide, goserelin, triptorelin, histrelin, degarelix, abiraterone, ketoconazole, flutamide, bicalutamide, nilutamide, enzalutamide, apalutamide, darolutamide, and bicalutamide.

Embodiment 79. A method for treating a mammal having CRPC, wherein said method comprises:
(a) administering an agent that can inhibit HER2/HER3 heterodimerization within said mammal; and
(b) administering an anti-androgen agent to said mammal.

Embodiment 80. The method of embodiment 79, wherein said mammal is a human.

Embodiment 81. The method of any one of embodiments 79-80, wherein said agent can induce HER3 homodimerization.

Embodiment 82. The method of any one of embodiments 79-80, wherein said agent is selected from the group consisting of trastuzumab, ARRY-380, erlotinib, gefitinib, afatinib, neratinib, and pertuzumab.

Embodiment 83. The method of any one of embodiments 79-82, wherein said CRPC is a metastatic CRPC.

Embodiment 84. The method of any one of embodiments 79-83, wherein said anti-androgen agent is selected from the group consisting of leuprolide, goserelin, triptorelin, histrelin, degarelix, abiraterone, ketoconazole, flutamide, bicalutamide, nilutamide, enzalutamide, apalutamide, darolutamide, and bicalutamide.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-9. (canceled)

10. A method for restoring androgen sensitivity to a CRPC within a mammal, wherein said method comprises subjecting said mammal to a therapy that reduces a systemic level of a NRG-1 polypeptide within said mammal.

11. The method of claim 10, wherein said therapy is selected from the group consisting of metastases-directed stereotactic body radiotherapy (SBRT) and therapeutic plasma exchange (TPE).

12. A method for restoring androgen sensitivity to a CRPC within a mammal, wherein said method comprises administering an inhibitor of a NRG-1 polypeptide to said mammal.

13. (canceled)

14. The method of claim 12, wherein said inhibitor of said NRG-1 polypeptide is selected from the group consisting of rucaparib, olaparib, sorafenib, and TAPI-2.

15. A method for restoring androgen sensitivity to a CRPC within a mammal, wherein said method comprises administering an inhibitor of a disintegrin and metalloproteinase domain-containing protein (ADAM) 10 polypeptide to said mammal.

16. (canceled)

17. The method of claim 15, wherein said inhibitor of said NRG-1 polypeptide is selected from the group consisting of INCB8765, GI 254023X, TAPI-0, and TAPI-2.

18. A method for restoring androgen sensitivity to a CRPC within a mammal, wherein said method comprises administering an inhibitor of an ADAM17 polypeptide to said mammal.

19. (canceled)

20. The method of claim 18, wherein said inhibitor of said NRG-1 polypeptide is selected from the group consisting of an anti-ADAM17 D1(A12) antibody, TAPI-0, and TAPI-2.

21. A method for restoring androgen sensitivity to a CRPC within a mammal, wherein said method comprises administering an inhibitor of a poly (ADP-ribose) polymerase (PARP) polypeptide to said mammal.

22. (canceled)

23. The method of claim 21, wherein said inhibitor of said NRG-1 polypeptide is selected from the group consisting of olaparib, rucaparib, niraparib, and talazoparib.

24. (canceled)

25. A method for restoring androgen sensitivity to a CRPC within a mammal, wherein said method comprises administering an agent that can inhibit heterodimerization of a human epidermal growth factor receptor (HER) 2 polypeptide and a HER3 polypeptide (HER2/HER3 heterodimerization) within said mammal.

26. The method of claim 25, wherein said agent can induce homodimerization of two HER3 polypeptides (HER3 homodimerization).

27. The method of claim 25, wherein said agent is selected from the group consisting of trastuzumab, ARRY-380, erlotinib, gefitinib, afatinib, neratinib, and pertuzumab.

28. (canceled)

29. (canceled)

30. A method for treating a mammal having CRPC, wherein said method comprises:
(a) subjecting said mammal to a therapy that can reduce a systemic level of NRG-1 polypeptides within said mammal; and
(b) administering an anti-androgen agent to said mammal.

31. The method of claim 30, wherein said therapy is selected from the group consisting of metastases-directed SBRT and TPE.

32. The method of claim 30, wherein said anti-androgen agent is selected from the group consisting of leuprolide, goserelin, triptorelin, histrelin, degarelix, abiraterone, ketoconazole, flutamide, bicalutamide, nilutamide, enzalutamide, apalutamide, darolutamide, and bicalutamide.

33. A method for treating a mammal having CRPC, wherein said method comprises:
(a) administering an inhibitor of a NRG-1 polypeptide to said mammal; and
(b) administering an anti-androgen agent to said mammal.

34. (canceled)

35. The method of claim 33, wherein said inhibitor of said NRG-1 polypeptide is selected from the group consisting of rucaparib, olaparib, sorafenib, and TAPI-2.

36. The method of claim 33, wherein said anti-androgen agent is selected from the group consisting of leuprolide, goserelin, triptorelin, histrelin, degarelix, abiraterone, ketoconazole, flutamide, bicalutamide, nilutamide, enzalutamide, apalutamide, darolutamide, and bicalutamide.

37. A method for treating a mammal having CRPC, wherein said method comprises:
(a) administering an inhibitor of an ADAM10 polypeptide to said mammal; and
(b) administering an anti-androgen agent to said mammal.

38. (canceled)

39. The method of claim 37, wherein said inhibitor of said ADAM10 polypeptide is selected from the group consisting of INCB8765, GI 254023X, TAPI-0, and TAPI-2.

40. The method of claim 37, wherein said anti-androgen agent is selected from the group consisting of leuprolide, goserelin, triptorelin, histrelin, degarelix, abiraterone, ketoconazole, flutamide, bicalutamide, nilutamide, enzalutamide, apalutamide, darolutamide, and bicalutamide.

41. A method for treating a mammal having CRPC, wherein said method comprises:
(a) administering an inhibitor of an ADAM17 polypeptide to said mammal; and
(b) administering an anti-androgen agent to said mammal.

42. (canceled)

43. The method of claim 41, wherein said inhibitor of said ADAM17 polypeptide is selected from the group consisting of an anti-ADAM17 D1(A12) antibody, TAPI-0, and TAPI-2.

44. The method of claim 41, wherein said anti-androgen agent is selected from the group consisting of leuprolide, goserelin, triptorelin, histrelin, degarelix, abiraterone, ketoconazole, flutamide, bicalutamide, nilutamide, enzalutamide, apalutamide, darolutamide, and bicalutamide.

45. A method for treating a mammal having CRPC, wherein said method comprises:
(a) administering an inhibitor of a PARP polypeptide to said mammal; and
(b) administering an anti-androgen agent to said mammal.

46. (canceled)

47. The method of claim 45, wherein said inhibitor of said PARP polypeptide is selected from the group consisting of olaparib, rucaparib, niraparib, and talazoparib.

48. The method of claim 45, wherein said anti-androgen agent is selected from the group consisting of leuprolide, goserelin, triptorelin, histrelin, degarelix, abiraterone, ketoconazole, flutamide, bicalutamide, nilutamide, enzalutamide, apalutamide, darolutamide, and bicalutamide.

49. A method for treating a mammal having CRPC, wherein said method comprises:
(a) administering an agent that can inhibit HER2/HER3 heterodimerization within said mammal; and
(b) administering an anti-androgen agent to said mammal.

50. The method of claim 49, wherein said agent can induce HER3 homodimerization.

51. The method of claim 49, wherein said agent is selected from the group consisting of trastuzumab, ARRY-380, erlotinib, gefitinib, afatinib, neratinib, and pertuzumab.

52. The method of claim 49, wherein said anti-androgen agent is selected from the group consisting of leuprolide, goserelin, triptorelin, histrelin, degarelix, abiraterone, ketoconazole, flutamide, bicalutamide, nilutamide, enzalutamide, apalutamide, darolutamide, and bicalutamide.

53. The method of claim 30, wherein said CRPC is a metastatic CRPC.

54. The method of claim 30, wherein said mammal is a human.
